(19) United States
(12) Patent Application Publication
(10) Pub. No.: US 2021/0324011 A1
(43) Pub. Date: Oct. 21, 2021

(54) SELF-ASSEMBLING PROTEIN HOMO-POLYMERS

(71) Applicant: UNIVERSITY OF WASHINGTON, Seattle, WA (US)

(72) Inventors: Hao SHEN, Seattle, WA (US); Jorge FALLAS, Seattle, WA (US); David BAKER, Seattle, WA (US)

(21) Appl. No.: 17/285,057

(22) PCT Filed: Oct. 24, 2019
(86) PCT No.: PCT/US2019/057768

§ 371 Date: Apr. 13, 2021

Related U.S. Application Data
(60) Provisional application No. 62/750,435, filed on Oct. 25, 2018.

(51) Int. Cl. C07K 14/00; C07K 1/00

(52) U.S. Cl. CPC C07K 14/001; C07K 2319/00; C07K 1/00

(57) ABSTRACT

Disclosed herein are polypeptides having the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO: 1-33 and 36, wherein the polypeptides include at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptides are capable of end-to-end homo-polymerization, homo-polymers of the polypeptides, and related capping and anchor proteins to facilitate homo-polymer formation.

### CROSS REFERENCE

This application claims priority to U.S. Provisional Patent Application Ser. No. 62/750,435 file Oct. 25, 2018, incorporated by reference herein in its entirety.

### FEDERAL FUNDING STATEMENT

This invention was made with government support under Grant No. W911NF-17-1-0318, awarded by the Defense Advanced Research Projects Agency. The government has certain rights in the invention.

### BACKGROUND

Natural protein filaments differ considerably in their dynamic properties: some, like collagen, are relatively static with turnover rates in order of several weeks, while others, like cytoskeletal polymers, are dynamic—growing or disassembling in response to changing physiological conditions. The fraction of the total residue-residue interactions in the filament that are within (rather than between) the monomeric building blocks is generally higher for dynamic polymers; the monomers are usually independently folded structures rather than relatively extended polypeptides. The building blocks in most reversibly assembling filaments have no internal symmetry, and hence multiple designed interfaces may be needed to drive formation of the desired structure. The reduced symmetry also makes the sampling problem more challenging, as the space of possible filament geometries is extremely large.

### SUMMARY

In one aspect, the disclosure provides non-naturally occurring polypeptides comprising the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is capable of end-to-end homo-polymerization. In one embodiment, the polypeptide includes at least 80%, 85%, 90%, 91%, 92%, 93%, 94%, 95%, 96%, 97%, 98%, 99%, or 100% of the identified interface residues. In another embodiment, the polypeptide is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21. In another embodiment, amino acid substitutions relative to the reference amino acid sequence are conservative amino acid substitutions.

In a further aspect, the disclosure provides homo-polymers comprising 2, 3, 4, 5, 10, 15, 20, 25, 50, 75, 100, or more identical polypeptides according to any embodiment or combination of embodiments disclosed herein associated end-to-end. In one embodiment, the homo-polymer comprises a helical filament. In another embodiment, the homo-polymer is bound to a surface. In one embodiment, the homo-polymer is bound to the surface via interaction with an anchor protein of any embodiment or combination of embodiments disclosed herein.

In one aspect, the disclosure provides methods of making the homo-polymer of any embodiment or combination of embodiments disclosed herein, comprising mixing multiple copies of identical polypeptide of any embodiment or combination of embodiments disclosed herein under conditions that promote homo-polymerization of the proteins, including but not limited to the conditions disclosed herein. In one embodiment, homo-polymerization at one or both ends of the homo-polymer is capped by mixing the polypeptides of any embodiment or combination of embodiments disclosed herein with a corresponding capping protein of any embodiment or combination of embodiments disclosed herein.

In another aspect, the disclosure provides anchor proteins, comprising:
(a) an oligomeric protein of cyclic symmetry;
(b) an optional amino acid linker; and
(c) a polypeptide any embodiment or combination of embodiments disclosed herein or a cap protein of any embodiment or combination of embodiments disclosed herein, linked (covalently or non-covalently) to the oligomeric protein of cyclic symmetry. In one embodiment, the anchor protein further comprises a fluorescent tag and/or one or more binding domains to direct the anchor to a desired location. In another embodiment, the anchor protein comprises the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues. In another embodiment, the anchor protein includes at least 80%, 85%, 90%, 91%, 92%, 93%, 94%, 95%, 96%, 97%, 98%, 99%, or 100% of the identified interface residues.

In another aspect, the disclosure provides capping proteins comprising the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36, wherein the capping protein includes changes in at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the capping protein is not capable of end-to-end homo-polymerization. In one embodiment, the capping protein comprises the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:37-40.

In another aspect, the disclosure provides recombinant nucleic acids encoding the polypeptide or protein of any embodiment or combination of embodiments disclosed herein. In another aspect, the disclosure provides expression vectors comprising the nucleic acid of any embodiment or combination of embodiments disclosed herein operatively linked to a promoter. In a further aspect, the disclosure provides recombinant host cell comprising the expression vector and/or nucleic acids disclosed herein.

In a further aspect, the disclosure provides methods for computational design of polypeptides capable of end-to-end homo-polymerization to form self-assembling helical filaments, comprising the steps described herein.

### DESCRIPTION OF THE FIGURES

FIG. 1. Filament architectures and computational design protocol. (A) The fraction of total residue-residue interactions within (rather than between) monomers. (B) Super helical parameters. (C) Computational design protocol. In (A) and (B) the properties of filaments generated by the design protocol are compared to those of naturally occurring proteins.

FIG. 2. CryoEM structure determination. Computational model (first panel), representative filaments in cryoEM micrographs (second panel), cryoEM structure (third panel) and overlay between model and structure (fourth panel) for (A) DHF58-r.m.s.d. 3.3 Å (B) DHF119-r.m.s.d. 2.3 Å (C) DHF91-r.m.s.d. 1.2 Å (D) DHF46-r.m.s.d. 2.2 Å (E) DHF79-r.m.s.d. 4 Å (F) DHF38-r.m.s.d. 0.9 Å (G) The high-resolution structure of design DHF119 is very close to the design model. Close up views of the two main intermonomer interfaces in the filament, with the computational model and cryoEM structure in sticks in the helical reconstruction density (3.4 Å resolution).

FIG. 3. Modular tuning of fiber diameter. DHF58 filament variants with different numbers of repeats were characterized by electron microscopy. (A) Top: number of repeats. Cross sections (middle) and side views of computational models based on the 4-repeat cryoEM structure. (B) Negative stain electron micrographs. (C) 2D class averages.

FIG. 4. Characterization of fiber growth and disassembly. (A) Construction of fiber anchors holding monomers in rigid body arrangement found in the filament. (B) Kinetics of DHF119-YFP filament assembly in vitro on glass surface coated with DHF119_C6 anchor. In right panel, the glass surface was coated with the non-cognate DHF91 anchor. (C) DHF119 filaments emanating from DHF119_C6 anchor coated magnetic bead incubated with monomer; beads on right lack anchor.

FIG. 5. Computational Sampling of helical filaments containing a given protein dimer. Left, dimeric complex sampled during the slide into contact stage of the protocol. Middle, helical filament produced by repeatedly applying the fourth root or the transform defined in A (n=4−the two subunits that define the helical symmetry are not in contact). Right, helical filament produced by repeatedly applying the transform defined in the first panel (n=1−the two subunits that define the helical symmetry are in contact).

FIG. 6. Comparison of designs generated from de novo Designed Helical Repeat proteins (DHRs) and natural asymmetric proteins. (A) Scatter plot of Rosetta binding energy for main and secondary interfaces for designs generated from DHRs, natural asymmetric proteins (PDB ID: 1stn, 2bk9 and 5gh1) and structurally verified de novo Designed Helical Filaments (DHFs). (B) Top and (C) side views for an example fiber design model generated from Staphylococcal nuclease (PDB ID: 1stn) colored by chains.

FIG. 7. Negative stain EM of insoluble filaments. (A) DHF4 (B) DHF5 (C) DHF8 (D) DHF16 (E) DHF17 (F) DHF23 (G) DHF25 (H) DHF28 (I) DHF31 (J) DHF34 (K) DHF36 (L) DHF40 (M) DHF43 (N) DHF44 (0) DHF46 (P) DHF47 (Q) DHF49 (R) DHF50 (S) DHF77.

FIG. 8. Negative stain EM of soluble filaments. (A) DHF9 (B) DHF20 (C) DHF38 (D) DHF48 (E) DHF51 (F) DHF52 (G) DHF58 (H) DHF62 (I) DHF76 (J) DHF78 (K) DHF79 (L) DHF82 (M) DHF91 (N) DHF107 (0) DHF119

FIG. 9. Protease induced filament assembly using SUMO fusion constructs. (A) Design DHF38 before treatment with SUMO protease. (B) Design DHF38 after treatment with SUMO protease.

FIG. 10. Overlay for each individual interface and both interfaces to highlight the differences/similarities between the models and experimental structures. (A) DHF58 (B) DHF119 (C) DHF91 (D) DHF46 (E) DHF79 (F) DHF38

FIG. 11. Helical lattice plots comparing designed helical symmetry (open diamonds) to experimentally determined helical symmetry (closed circles) for (A) DHF58 (B) DHF119 (C) DHF91 (D) DHF46 (E) DHF79 and (F) DHF38.

FIG. 12. Filament assembly kinetics for DHF119. (A) Kinetic measurements of filament assembly by solution scattering. (B) Extrapolation of Critical concentration for assembly using the asymptotic values for the fits in (A).

FIG. 13. Concentration dependent assembly for DHF119. Top, Negative stain EM micrograph of DHF119 at 34.5 μM concentration in 25 mM Tris and 75 mM NaCl (left), 1M GuHCl (middle), 2M GuHCl (right). Bottom, Negative stain EM micrograph of DHF119 at 6.9 μM (left), 3.5 μM (middle), 0.7 μM (left) concentration in 25 mM Tris and 75 mM NaCl.

FIG. 14. Design of Anchoring Proteins. A library of designed oligomers with cyclic symmetry around the Z axis is aligned with a layer of fiber components taken from the cryoEM structure, with the helical axis also aligned along Z. Translations and rotations around Z are applied to find the closest distance between the oligomer termini and the fiber components. These are then linked using a flexible linker and substituting the fiber component for the appropriate capping accessory protein.

FIG. 15. Analysis of growth kinetics of DHF119 GFP fiber at 1804 concentration from biotinylated anchor proteins DHF119_C6 immobilized on streptavidin-coated slides monitored by TIRF microscopy over 30 minutes. (A) Tracked length over time for 3 individual fibers. (B) Histogram of linear-fit growth rate for 1000 tracked fibers (8.4 nm/minute on average with standard deviation of 7.2).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect, the disclosure provides non-naturally occurring polypeptides comprising the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is capable of end-to-end homo-polymerization. As used herein “wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues” means that at least the recited percentage of interface residues are not modified relative to the reference SEQ ID NO.

DHF40
(SEQ ID NO: 01)
SSEKEELRERLVKICVELAKLKGDDILKAAEAAEEAFRLVVLAAMLAGIDSSEVLELAIRLI
KTCVVLAAMEGYDISEACRAAAEAFTRVAMAALRAGITSSLVLKAAIELIKECVLNAAVEGY
DISEACRAAAEAFKRVAEAAKRAGITSLETLLRAIEEIRKRVEEAQREGNDISEACRQAAEE
FRKKAEELKRRGDV
DHF76
(SEQ ID NO: 02)
MGLEKEVLRVKLVKICVLLARLKGDDTEEAREAARQAFEVVRLAAELAGIDSSEVLELAIRL
IKECVENAQREGYDISLACMLAALAFKRVAEAAKRAGITSSEVLELAIRLIKECVENAQRDG
YDISEACRAAAEAFKRVAEAAKRAGITSSETLKRAIEEIRKRVEEAQREGNEISEACRQAAE
EFRKKAEELKRLE
DHF77
(SEQ ID NO: 03)
MGDSMKLVMLLLKKAVTLAKLNNDDMVALEIERAAKQIVLALAVNKSDEMAKVMLALAKAVL
LAAKNNDDEVAREIARAAAEIVEALKENNSDEMAKVMLALAKAVLLAAKNNDDEVAREIARA
AAEIVEALRENNSDEMAKKMLELAKRVLDAAKNNDDETAREIARQAAEEVEADRENLE
DHF17
(SEQ ID NO: 04)
(MGHHHHHH)SSGSSEKEELRERLVEIVVALAKAKGDDTELAREAAREAFELVREAAERAGI
DSSMVLILAIQLILEVVANAAKEGYDISLAALAAAEAFKRVAEAAKRAGITSSMVLILAIRL
IKEVVENAQREGYDISLAALAAAEAFKRVAEAAKRAGITSSMTLMRAIEEIRKRVEEAQREG
NDISEAARQAAEEFIIKAVVLKARGDV*
DHF23
(SEQ ID NO: 05)
MEKEVLREKLVKIVVEAAKLKGDDTLEAKLAAMEAYVLVVLAAELAGIDSSEVLELAIRLIK
QVVILAALEGYDISEATRATAEAFKTVAEAAKTAGITSSEVLELTIRLIKEVVENAQREGYD
ISEAARAAAEAFKRVAEAAKRAGITSSETLKRAIEEIRKRVEEAQREGNDISEAARQAAEEF
RKKAEELKRRGLE
DHF36
(SEQ ID NO: 06)
MVEKNMLRLRLVKIVVENAKTKGDDTEEAREAAREAFEKVRVAAEVAGIDSSEVLELAIRLI
KEVVLLALLEGYDISEAARAAAEAFVRVAVAAKIAGITSSEVLELAIRLIKEVVMNAMMEGY
DISEAARAAAEAFKLVAEAAKRAGITSSETLKRAIEEIRKRVEEAQREGNNISVAALEAADE
FRKKAEELKRRGLE
DHF43
(SEQ ID NO: 07)
(MGHHHHHH)SSGNDEKEKLKELLKRAEELAKSPDPEDLKEAVRLAEEVVRERPGSNLAKKA
LLIILRAAVELAALPDPEALKEAVKAAEKVVREQPGSSLARLALEIILMAAEELAKLPDPEA
LKEAVKAAEKVVREQPGSNLAKKALEIIERAAMMLKLSPDPEAQVEAMRAELKVVEER*
DHF46
(SEQ ID NO: 08)
(MGHHHHHH)SSGTKEERVLLMKVAILAIVAAKKGNIDEVRKALELALLIAKVSGTTEAVKL
ALEVVARVAIEAARRGNIDAVREALEVALEIARESGTTEAVKLALEVVARVAIEAARRGNTE
AVVEALLVALEIAKESGTEEAVRLALEVVKRVSNEALKQGNVDAVKVALEVRKMIEELSG
DHF52
(SEQ ID NO: 09)
SLKELLELERVAVEAIVAAAEGNTMEVIEQLQRAMLIAVLSGTTSAVKLALEVVARVAIEAA
RIGNIDAVRIALSVALTIAVISGTTEAVKLALEVVARVAIEAARRGNIDAVREALEVALEIA
RESGTEEAVRLALEVVKRVSDEAKKQGNEDAVKEAEEVRKKIEEESG
DHF91
(SEQ ID NO: 10)
MGDERRELEKVAVKAIMAAMLGNIDEVREQLQRALEIARESGTLLAVVLALEVVARVAIEAA
RKGNIDAVREALEVALEIARESGTKVAVVLALEVVARVAIEAARRGNVLAVILALEVALEIA
RESGTEEAALLAVEVVVRVSDEAKKQGNAVAVAVAEQVAKKILE
DHF5
(SEQ ID NO: 11)
ALDLLARVIVLVMEAVKLLVLAVIMGLEMILRVALRLAEEAARAAKAVLLLAEVEGDPEVAL
KAVELVVRVAELLLRIAKESGSEEALERALRVAEEAARLAKRVLELAEKQGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREG
DHF8
(SEQ ID NO: 12)
MGLELSLVINLVLLANLLHLQAMMKGSSEDLEKALRTAEEAAREAKKVLEQAEKNGDPEVAL
RAVELVVIVAITLLLIAISSGSEEALERALRVAEEAARLAKRVLELAEKAGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREGLE
DHF25
(SEQ ID NO: 13)
ARVVLKRVEKLVKEAETLLLVAIMKGSEEDLEKALRTAEEAAREAKKVLVLAVVVGDPEVAL
RAVELVVRVAELLLLIAIISGSEEALERALRVAEEAARLAKRVLELAENQGDPEVALRAVEL
VVRVAELLLMIAKVSGSEEALERALRVAEEAARLAKRVLELAEKQGDPEVARRAVELVKRVA
ELLERIARESGSEEAKERAERVREEALQLQARVMNLREREG
DHF31
(SEQ ID NO: 36)
AAIETLNVMTLVMLALLLLIQAISKGSAEDLEKALRTAEEAAREAKKVLEQAEKEGDPDQAL
KAVELVVVVAQLLLQIAIASGSREALERALRVAEEAARLAKRVLELAEKQGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREG
DHF38
(SEQ ID NO: 14)
AEELLKRVEKLVKEAEELLRQAMKKGSEELLEVALVVAQMAAREAKKVLTMAEVEGDPEVAL
RAVELVVRVAELLLRIALVSGSEEALERALRVAEEAARLAKRVLELAESQGDPEVALRAVEL
VVRVAELLLLIAKVSGSEEALERALRVAEEAARLAKRVLELAEKQGDPAVAILAVMLVKRVA
ELLENIARETGSETAKERAERVREEARELQERVKELKERAG
DHF44
(SEQ ID NO: 15)
AVLALMVVNLLVQLAIELLSRAMLKGSAEDLEKALRTAEEAAREAKKVLEQAEKDGDPEVAL
LAVELVVVVATILLMIAKISGSEEALERALRVAEEAARLAKRVLELAEKQGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREG
DHF48
(SEQ ID NO: 16)
MAVEEAIRVMRLVREAEQVLLQAKMMGSERVLEMALRTAEEAAREAKLVLAVAELEGDPVVA
LIAVELVVRVAELLLRIAKESGSEEALERALRVAEEAARLAKRVLELAEKQGDPEVALRAVE
LVVRVAELLLRIAKESGSEEALERALRVAEEAARLAKRVLELAEKQGDPEVARRAVELVKRV
AELLERIARESGSEEAKERAERVREEARELQERVKELREREGLE
DHF50
(SEQ ID NO: 17)
AIVQLMTVIAMAVIANALLIRAIVNGSAEDLEKALRTAEEAAREAKKVLEQAEKDGDPEVAL
RAVELVVLVAVNLLLIAKESGSEEALERALRVAEEAARLAKRVLELAEKVGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREG
DHF51
(SEQ ID NO: 18)
AVDQMIRVIVLVKEAEEALLNAKLLGSEMALTLALRTAEEAARVAKTVLELAEKDGDPVVAL
LAVELVVRVAELLLRIAKESGSEEALERALRVAEEAARLAKRVLELAEKQGDPEVALRAVEL
VVRVAELLLRIAKESGSEEALERALRVAEEAARLAKRVLELAEKQGDPEVARRAVELVKRVA
ELLERIARESGSEEAKERAERVREEARELQERVKELREREG
DHF58
(SEQ ID NO: 19)
MELLRVVMLVKEAEELLTLAVIKGSEDDLQKALRTAVEAAREAVKVLLQAVKRGDPEVALRA
VELVVRVAELLLRIAKESGSELALKMALLVAEEAARLAKIVLELAEKQGDPEVALRAVELVV
RVAELLLRIAKESGSEEALERALRVAEEAARLAKRVLELAEKQGDPEVARRAVELVKRVAEL
LERIARESGSEEAKERAERVREEARELQERVKELREREGLE
DHF79
(SEQ ID NO: 20)
MGPEDELKRVEKLVKEAEMLLMLAKIKGSEKDLEKALRTAEEAAREAKKVLEQAEKEGDPEV
ALRAVELVVRVAELLLRIAKESGSEEALMTALLVAVVAATLAVRVLVLAAVQGDPEVALRAV
ELVVRVAELLLRIAKESGSEEALERALRVAEETARLAKRVLELAQEQGDPVVAIAAVMLVKR
VAELLERIARESGSEEAKERAERVREEARELQERVKELREREGLE
DHF119
(SEQ ID NO: 21)
MGPEDELKRVEKLVKEAEALLIVAKIKGSKRDLEKALRTAEEAAREAVKVLVQALLEGDPEV
ALRAVELVVRVAELLLRIAKESGSREALLRALIVAEEAAKLAKMVLELAEKQGDPEVALRAV
ELVVRVAELLLRIAKESGSEEALERALRVAEEAARLAKRVLELAEEQGDPLVAKMAVELVKR
VAELLERIARESGSEEAKERAERVREEARELQERVKELREREGLE
DHF9
(SEQ ID NO: 22)
MGLILELAKLSLERARLASEAGDRVEFKIAAEKALLLAKVLVLQAKKEGDPELVLEAAKVAL
RVAELAAKNGDKEVFKKAAESALEVAKRLVEVASKEGDPELVLEAAKVALRVAELAAKNGDK
EVFKKAAESALEVAKRLVEVASKEGDPELVEEAAKVAEEVRKLAKKQGDEEVYEKARETARE
VKEELKRVREEKGDLE
DHF16
(SEQ ID NO: 23)
MGQILMKAKLSLVKAAEASLSGDELEFRIAAETALVLAKLLVLQAKKKGDPELVLEAAKVAL
RVAELAAKNGDKEVFKKAAESALEVAKRLVEVASKEGDPELVLEAAKVALRVAELAAKNGDK
EVFKKAAESALEVAKRLVEVASKEGDPELVEEAAKVAEEVRKLAKKQGDEEVYEKARETARE
VKEELKRVREEKGLE
DHF34
(SEQ ID NO: 24)
AEVILLVAVLLLAKAVEAVLMGNRVAFRLAAELALRVAKLLVIMAKVEGDPELVLEAAKVAL
VVASLAALNGDKEVFKKAAESALEVAKRLVEVASKEGDPELVLEAAKVALKVAELAAKNGDK
EVFKKAAESALEVAKRLVEVASKEGDPELVEEAAKVAEEVRKLAKKQGDEEVYEKARETARE
VKEELKRVREEKG
DHF78
(SEQ ID NO: 25)
MGELVLLVAKLALAVAVQASALGDEEVFRKAAEKALELAKRLVELAKLIGDPELVLEAAKVA
LEVAELAAKNGDKEVFKKAAESALEVAKRLVEVASKEGDPELVLEAAKVALRVAELAAKNGD
KEVFKKAAESALEVAKRLVEVASKEGDPELVEEAAKVAEEVRKLAKKQGDEEVYEKARETAR
EVKEELKRVREEKGLE
DHF82
(SEQ ID NO: 26)
MGPEEILESAKESLERAREASEAGLELVFRLAAEVALELAKRLVEQAKKEGDPELVLEAAKV
ALRVAELAAKNGDKEVFKKAAESALEVAKRLVEVASKEGDPELVLEAAKVALRVAELAAKNG
DKEVEKKAAESALEVAKRLVEVASKEGDPTLVIMALIVAEEVIKLASKQGDELVMLKAVETA
KEVKEEAMRVMLEKGLE
DHF107
(SEQ ID NO: 27)
MGPEEILERAKESLERAREASERGDEMEFRIAAQKALELARQLVLQALAEGDPELVLEAAKV
ALRVAELAAKNGDKEVEKKAAESALEVAKVLVLVASKEGKPELVLEAAKVALRVAVLAAMNG
DKEVEKKAAESALEVAKRLVEVASKEGDPELVLEAAKVAEKVRVLAAVQGDEEVYEKARETA
REVKEELKRVMEEKGLE
DHF28
(SEQ ID NO: 28)
MQLMTAILMAILVATMVELVSNRALLEGNPDLVVSANELRRAVEEAIEEAKKQGNPELVMWV
AIAAKVAAEVIKVAIQAELEGNRDLFRAALELVRAVIEAIEEAVKQGNPELVEWVARAAKVA
AEVIKVAIQAEKEGNRDLFRAALELVRAVIEAIEEAVKQGNPELVERVARLAKKAAELIKRA
IRAEKEGNRDERREALERVREVIERIEELVRQG
DHF47
(SEQ ID NO: 29)
INLTIAILVAILVATMVELVAIRAQLEGNPELAASAMELRRAVEEAIEEAEKQGNPLLVLWV
AVAAKVAAEVIKVAIQAEKEGNRDLFRAALELVRAVIEAIEEAVKQGNPELVEWVARAAKVA
AEVIKVAIQAEKEGNRDLFRAALELVRAVIEAIEEAVKQGNPELVERVARLAKKAAELIKRA
IRAEKEGNRDERREALERVREVIERIEELVRQGN
DHF4
(SEQ ID NO: 30)
ADEELARILIEMAKTAAEAAQMAAEVTGDPRVRLLAVRLKMLALMAALEVILDPSSSDVNEA
LKLIVEAIEAAVKALLAAVRTGDPEVRELARELVRLAVEAAEEVQRNPSSSDVNEALKLIVE
AIEAAVRALEAAERTGDPEVRELARELVRLAVEAAEEVQRNPSSEEVNEALKKIVKAIQEAV
ESLREAEESGDPEKREKARERVREAVERAEEVQRN
DHF20
(SEQ ID NO: 31)
MDEMKARLLIERAKEAAERAQEAAERTGDPLVRLLAEMLKRLAQEAAEEVKRDPSSSDVNEA
LKLIVEAIEAAVRALEAAERTGDPEVRELASELVRLAVEAAEEVQRNPSSSDVNEALKLIVE
AIEAAVRALEAAERTGDPRVRELARELVRLAVEAAEEVQRNPSSVEVALALLKIVKAIEEAV
ESLREAEESGDPMKRLEAALRVREAVERAEEVQRN
DHF49
(SEQ ID NO: 32)
(MGHHHHHH)SSGSDEEEARELIERAAEAAKRAVEAAERTGDPNVVELAKELVRLAQEAAEE
VKRDPSSSDVNEALKLIVEAIEAAVRALEAAERTGDPVVRLLARELVRLAVEAAEEVQRNPS
SSDVNEALKLIVEAIEAAVRALEAAERTGDPEVRELARELVRLAVEAAEEVQRNPSSEQVNE
ALKKIVKAIQEAVESLREAEESGDPEKRDKARLRVLEAVIRAMVVQIN*
DHF62
(SEQ ID NO: 33)
MGIVLVIESLEAEVRLEKAKVLSVLARVRGDLKELAEALIEEARAVQELARVACEKGNSEEA
ERASEKAQRVLEEARKVSEEAREQGDDEVLALALIAIALAVLALAEVACCRGNSEEAERASE
KAQRVLEEARKVSEEAREQGDDEVLALALIAIALAVLALAEVACCRGNKEEAERAYEDARRV
EEEARKVKESAEEQGDSEVKRLAEEAEQLAREARRHVQECRGLE.

The polypeptides of this aspect can be used, for example, as monomers for the assembly of homo-polymeric filaments. As disclosed herein, the inventors developed a general computational approach to designing self-assembling helical filaments from monomeric polypeptides, and use it to design polypeptides of the disclosure that can assemble into micron scale, homo-polymeric helical filaments with a wide range of geometries in vivo and in vitro. The polypeptides are idealized repeat proteins, and hence the diameter of the filaments can be systematically tuned by varying the number of repeat units.

The polypeptides are “non-naturally occurring” in that the entire polypeptide is not found in any naturally occurring polypeptide. The “identified interface residues” are those residues that are in bold-font and underlined in the sequences shown herein. As shown in the examples that follow, the polypeptides can undergo significant modification in their primary amino acid sequence (particularly in non-interface residues) while retaining the ability to homo-polymerize.

In one embodiment, the polypeptide includes at least 80%, 85%, 90%, 91%, 92%, 93%, 94%, 95%, 96%, 97%, 98%, 99%, or 100% of the identified interface residues. In a specific embodiment, the polypeptide includes at least 90%, 91%, 92%, 93%, 94%, 95%, 96%, 97%, 98%, 99%, or 100% of the identified interface residues. In another specific embodiment, the polypeptide includes at least 95%, 96%, 97%, 98%, 99%, or 100% of the identified interface residues. In a further specific embodiment, the polypeptide includes 100% of the identified interface residues.

In one specific embodiment, the polypeptide amino acid sequence is at least 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36. In another specific embodiment, the polypeptide amino acid sequence is at least 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36. In a further specific embodiment, the polypeptide amino acid sequence is at least 90%, 91%, 92%, 93%, 94%, 95%, 96%, 97%, 98%, 99%, or 100% identical to the full length of the amino acid sequence selected from the group consisting of SEQ ID NO:1-33 and 36.

In one specific embodiment, the polypeptide amino acid sequence is at least 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36 and includes at least 80%, 85%, 90%, 91%, 92%, 93%, 94%, 95%, 96%, 97%, 98%, 99%, or 100% of the identified interface residues. In another specific embodiment, the polypeptide amino acid sequence is at least 80%, 85%, 90%, 91%, 92%, 93%, 94%, 95%, 96%, 97%, 98%, 99%, or 100% identical to the full length of the amino acid sequence selected from the group consisting of SEQ ID NO:1-33 and 36 and includes at least 90%, 91%, 92%, 93%, 94%, 95%, 96%, 97%, 98%, 99%, or 100% of the identified interface residues. In a further specific embodiment, the polypeptide amino acid sequence is at least 90%, 91%, 92%, 93%, 94%, 95%, 96%, 97%, 98%, 99%, or 100% identical to the full length of the amino acid sequence selected from the group consisting of SEQ ID NO:1-33 and 36 and includes at least 95%, 96%, 97%, 98%, 99%, or 100% of the identified interface residues.

In another embodiment, the polypeptide is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21. In one specific embodiment, the polypeptide is at least 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21. In another specific embodiment, the polypeptide is at least 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21. In a further specific embodiment, the polypeptide is at least 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21.

In one specific embodiment, the polypeptide is at least 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21, and includes at least 80%, 85%, 90%, 91%, 92%, 93%, 94%, 95%, 96%, 97%, 98%, 99%, or 100% of the identified interface residues. In another specific embodiment, the polypeptide is at least 80%, 85%, 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21 and includes at least 90%, 91%, 92%, 93%, 94%, 95%, 96%, 97%, 98%, 99%, or 100% of the identified interface residues. In a further specific embodiment, the polypeptide is at least 90%, 91%, 92%, 93%, 94%, 95%, 96%, 97%, 98%, 99%, or 100% identical to the full length of the amino acid sequence selected from the group consisting of SEQ ID NOs:8, 10, 14, and 19-21 and includes at least 95%, 96%, 97%, 98%, 99%, or 100% of the identified interface residues.

In a further aspect, the disclosure provides homo-polymers comprising 2, 3, 4, 5, 10, 15, 20, 25, 50, 75, 100, or more identical polypeptides according to any embodiment or combination of embodiments disclosed herein associated end-to-end. As disclosed in the examples that follow, the polypeptides are idealized repeat proteins, and hence the diameter of the resulting homo-polymers can be systematically tuned by varying the number of polypeptide units. The assembly and disassembly of the homo-polymers (also referred to herein as filaments) can be controlled, for example by engineered anchor and capping proteins built from polypeptide monomers lacking one of the interaction surfaces as discussed in more detail herein. The highly ordered homo-polymeric structures can be used, for example, in fabrication of new multi-scale metamaterials.

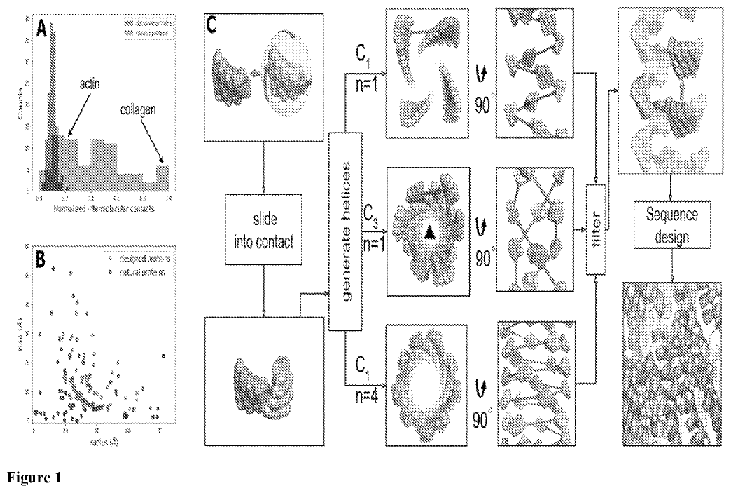

In one embodiment, the homo-polymer comprises a helical filament. The examples provide detailed discussion of how the polypeptide monomers were designed to assemble into helical homo-polymers. The resulting polypeptides designs span the range of helical parameters (diameter, rise, and rotation); see Table 1 and FIG. 1. In another embodiment, the homo-polymer is bound to a surface. The surface may be any suitable surface for an intended use, including but not limited to glass, plastic, polysaccharides, nylon, nitrocellulose, Teflon, microtiter plates, membranes, beads, etc. In one embodiment, the homo-polymer is bound to the surface via interaction with an anchor protein of any embodiment or combination of embodiments as disclosed in detail herein.

In another aspect the disclosure provides capping proteins comprising the amino acid sequence that is at least 50%, 55%, 60%, 65%, 70%, 75%, 80%, 85%, 90%, 91%, 92%, 93%, 94%, 95%, or more identical to the full length of the amino acid sequence selected from the group consisting of SEQ ID NO:1-33 and 36, wherein the polypeptide includes changes in at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is not capable of end-to-end homo-polymerization. Thus, the capping proteins are closely related to the polypeptides of the disclosure but are modified to eliminate the ability to homo-polymerize at one end of the protein.

In one specific embodiment, the capping protein amino acid sequence is at least 70%, 75%, 80%, 85%, 90%, 91%, 92%, 93%, 94%, 95%, or more identical to the full length of the amino acid sequence selected from the group consisting of SEQ ID NO:1-33 and 36. In another specific embodiment, the capping protein amino acid sequence is at least 80%, 85%, 90%, 91%, 92%, 93%, 94%, 95% or more identical to the full length of the amino acid sequence selected from the group consisting of SEQ ID NO:1-33 and 36. In a further specific embodiment, the capping protein amino acid sequence is at least 90%, 91%, 92%, 93%, 94%, 95%, or more identical to the full length of the amino acid sequence selected from the group consisting of SEQ ID NO:1-33 and 36.

In another embodiment, the capping protein is at least 50%, 55%, 60%, 65%, 70%, 75%, 80%, 85%, 90%, 91%, 92%, 93%, 94%, 95%, or more identical to the full length of the amino acid sequence selected from the group consisting of SEQ ID NOs:8, 10, 14, and 19-21. In one specific embodiment, the capping protein is at least 70%, 75%, 80%, 85%, 90%, 91%, 92%, 93%, 94%, 95%, or more identical to the full length of the amino acid sequence selected from the group consisting of SEQ ID NOs:8, 10, 14, and 19-21. In another specific embodiment, the capping protein is at least 80%, 85%, 90%, 91%, 92%, 93%, 94%, 95%, or more identical to the full length of the amino acid sequence selected from the group consisting of SEQ ID NOs:8, 10, 14, and 19-21. In a further specific embodiment, the capping protein is at least 90%, 91%, 92%, 93%, 94%, 95%, or more identical to the full length of the amino acid sequence selected from the group consisting of SEQ ID NOs:8, 10, 14, and 19-21.

In one embodiment, the capping protein comprises the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:37-40.

DHF58_N_cap
(SEQ ID NO: 37)
MGPEDELKRVEKLVKEAEELLTLAVIKGSEDDLQKALRTAVEAAREAKKV
LEQAEKEGDPEVALRAVELVVRVAELLLRIAKESGSELALKMALLVAEEA
ARLAKRVLELAEKQGDPEVALRAVELVVRVAELLLRIAKESGSEEALERA
LRVAEEAARLAKRVLELAEKQGDPEVARRAVELVKRVAELLERIARESGS
EEAKERAERVREEARELQERVKELREREGLE
DHF58_C_cap
(SEQ ID NO: 38)
MGELLRVVMLVKEAEELLRQAKEKGSEEDLEKALRTAEEAAREAVKVLLQ
AVKRGDPEVALRAVELVVRVAELLLRIAKESGSEEALERALRVAEEAARL
AKIVLELAEKQGDPEVALRAVELVVRVAELLLRIAKESGSEEALERALRV
AEEAARLAKRVLELAEKQGDPEVARRAVELVKRVAELLERIARESGSEEA
KERAERVREEARELQERVKELREREGLE
DHF119_N_cap
(SEQ ID NO: 39)
MGPEDELKRVEKLVKEAEELLRQAKEKGSEEDLEKALRTAEEAAREAVKV
LVQALLEGDPEVALRAVELVVRVAELLLRIAKESGSREALLRALIVAEEA
AKLAKMVLELAEKQGDPEVALRAVELVVRVAELLLRIAKESGSEEALERA
LRVAEEAARLAKRVLELAEEQGDPEVARRAVELVKRVAELLERIARESGS
EEAKERAERVREEARELQERVKELREREGLE
DHF119_C_cap
(SEQ ID NO: 40)
MGPEDELKRVEKLVKEAEALLIVAKIKGSKRDLEKALRTAEEAAREAKKV
LEQAEKEGDPEVALRAVELVVRVAELLLRIAKESGSEEALERALRVAEEA
ARLAKRVLELAEKQGDPEVALRAVELVVRVAELLLRIAKESGSEEALERA
LRVAEEAARLAKRVLELAEKQGDPLVAKMAVELVKRVAELLERIARESGS
EEAKERAERVREEARELQERVKELREREGLE

In one embodiment, the capping protein is at least 50%, 55%, 60%, 65%, 70%, 75%, 80%, 85%, 90%, 91%, 92%, 93%, 94%, 95%, 96%, 97%, 98%, 99%, or 100% identical at the identified interface residues of SEQ ID NOs:37-40.

In another aspect, the disclosure provides methods of making the homo-polymer of any embodiment or combination of embodiments disclosed herein, comprising mixing multiple copies of identical polypeptide of any embodiment or combination of embodiments disclosed herein under conditions that promote homo-polymerization of the proteins, including but not limited to the conditions disclosed in the examples that follow. In one embodiment, homo-polymerization at one or both ends of the homo-polymer is capped by mixing the polypeptides of any embodiment or combination of embodiments disclosed herein with a corresponding capping protein of any embodiment or combination of embodiments disclosed herein. The “corresponding” capping protein is one with the same name/designation as the polypeptides of SEQ ID NO:1-33 and 36, but modified to eliminate the ability to homo-polymerize at one or both ends of the protein.

In another aspect, the disclosure provides anchor proteins, comprising:
(a) an oligomeric protein of cyclic symmetry;
(b) an optional amino acid linker; and
(c) a polypeptide any embodiment or combination of embodiments disclosed herein or a capping protein of any embodiment or combination of embodiments disclosed herein, linked (covalently or non-covalently) to the oligomeric protein of cyclic symmetry.

The anchor proteins can be used, for example, to anchor the homo-polymers to a surface and to direct assembly of homo-polymer from a surface.

Any suitable oligomeric protein of cyclic symmetry may be used in the anchor proteins of the disclosure. The oligomeric protein of cyclic symmetry should arrange monomers in close approximation of geometry as in the designed filament structure. Exemplary oligomeric proteins of cyclic symmetry include, but are not limited to, those described in published PCT application WO2017/173356 and published US Application US-20190155988, each incorporated by reference herein in its entirety.

Any suitable amino acid linker may be used as deemed appropriate for an intended use, including but not limited to Gly-Ser rich linkers.

In one embodiment, the anchor protein further comprises a fluorescent tag and/or one or more binding domains to direct the anchor to a desired location.

In another embodiment, the anchor protein comprises a polypeptide that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues.

DHF119_C6_anchor
(SEQ ID NO: 34)
MGGGLNDIFEAQKIEWHEGGSGGSGGSGGSTETLIRLLEELARVLLEILKQNGVPTNVIEAV
RKAMEILLKMLKNSDNTAEAAAYMAIAMILLLILAKGGSGGSGGSGGSGGSSPEDELKRVEK
LVKEAEALLIVAKIKGSKRDLEKALRTAEEAAREAKKVLEQAEKEGDPEVALRAVELVVRVA
ELLLRIAKESGSREALLRALIVAEEAARLAKRVLELAEKQGDPEVALRAVELVVRVAELLLR
IAKESGSEEALERALRVAEEAARLAKRVLELAEEQGDPLVAKMAVELVKRVAELLERIARES
GSEEAKERAERVREEARELQERVKELREREGLE
DHF119_C6_GFP_anchor
(SEQ ID NO: 35)
MGGGLNDIFEAQKIEWHEGGSGGSGGSGGSTETLIRLLEELARVLLEILKQNGVPINVIEAV
RKAMEILLKMLKNSDNTAEAAAYMAIAMILLLILAKGGSGGSGGSGGSGGSSPEDELKRVEK
LVKEAEALLIVAKIKGSKRDLEKALRTAEEAAREAVKVLVQALLEGDPEVALRAVELVVRVA
ELLLRIAKESGSREALLRALIVAEEAAKLAKMVLELAEKQGDPEVALRAVELVVRVAELLLR
IAKESGSEEALERALRVAEEAARLAKRVLELAEEQGDPLVAKMAVELVKRVAELLERIARES
GSEEAKERAERVREEARELQERVKELREREG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Italicized portion is the GS linker and super-folded green
fluorescent protein.

In one embodiment, the anchor protein includes at least 80%, 85%, 90%, 91%, 92%, 93%, 94%, 95%, 96%, 97%, 98%, 99%, or 100% of the identified interface residues. In another embodiment, the anchor protein comprises a polypeptide that is at least 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 80%, 85%, 90%, 91%, 92%, 93%, 94%, 95%, 96%, 97%, 98%, 99%, or 100% of the identified interface residues. In a further embodiment, the anchor protein comprises a polypeptide that is at least 80%, 85%, 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 90%, 91%, 92%, 93%, 94%, 95%, 96%, 97%, 98%, 99%, or 100% of the identified interface residues. In one embodiment, the anchor protein comprises a polypeptide that is at least 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 95%, 96%, 97%, 98%, 99%, or 100% of the identified interface residues.

As used throughout the present application, the term “polypeptide” or “protein” is used in its broadest sense to refer to a sequence of subunit amino acids. The polypeptides of the invention may comprise L-amino acids+glycine, D-amino acids+glycine (which are resistant to L-amino acid-specific proteases in vivo), or a combination of D- and L-amino acids+glycine. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another embodiment, amino acid substitutions relative to the reference amino acid sequence are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides or proteins comprising conservative amino acid substitutions can be tested in any one of the assays described herein to confirm that a desired activity, e.g. homo-polymerization capability, is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The polypeptides, capping proteins, or anchor proteins of the disclosure may include additional residues at the N-terminus, C-terminus, or a combination thereof; these additional residues are not included in determining the percent identity of the polypeptides or proteins of the invention relative to the reference polypeptide. Such residues may be any residues suitable for an intended use, including but not limited to tags. As used herein, “tags” include general detectable moieties (i.e.: fluorescent proteins, antibody epitope tags, etc.), therapeutic agents, purification tags (His tags, etc.), linkers, ligands suitable for purposes of purification, ligands to drive localization of the polypeptide, peptide domains that add functionality to the polypeptides, etc.

In a further aspect the disclosure provides nucleic acids encoding the polypeptide or protein of any embodiment or combination of embodiments of each aspect disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operatively linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In one aspect, the disclosure provides host cells that comprise the nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further aspect, the disclosure provides methods for computational design of polypeptides capable of end-to-end homo-polymerization to form self-assembling helical filaments, comprising the steps described herein.

### EXAMPLES

Summary: We describe a general computational approach to designing self-assembling helical filaments from monomeric proteins, and use it to design proteins that assemble into micron scale helical filaments with a wide range of geometries in vivo and in vitro. CryoEM structures of six designs are close to the computational design models. The filament building blocks are idealized repeat proteins, and hence the diameter of the filaments can be systematically tuned by varying the number of repeat units. The assembly and disassembly of the filaments can be controlled by engineered anchor and capping units built from monomers lacking one of the interaction surfaces. The ability to generate dynamic highly ordered structures that span micrometers from protein monomers opens up possibilities for the fabrication of new multi-scale metamaterials.

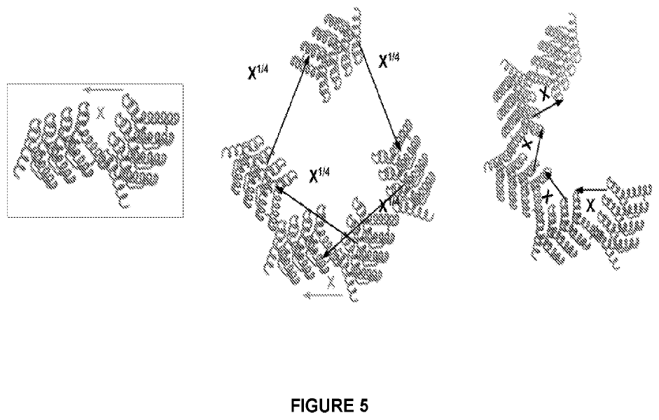

To tackle the challenge of de novo designing dynamic protein filaments, we devised a computational approach that exploits multiple inter-monomer interfaces to reduce the size of the search space (FIG. 1). Simple helical symmetry results from repeated application of a single rigid body transform; we also consider architectures in which multiple such simple helical filaments are arrayed with cyclic symmetry. Hence the search is over the 6 rigid body degrees of freedom, and the discrete degrees of freedom associated with the different cyclic symmetries. The approach starts from an arbitrary asymmetric protein monomer structure, and generates a second randomly oriented copy in physical contact by 1) applying a random rotation (3 degrees of freedom), 2) choosing a random direction (two degrees of freedom), and sliding the second copy towards the first until they come into contact (FIG. 1C, left; the sliding into contact effectively reduces the number of degrees of freedom from the six for an arbitrary rigid body transform to five). Successive monomers related by the filament defining rigid body transform need not themselves be in contact, and such arrangements are rare in biology. To go beyond this restriction, we considered not only filaments generated by the rigid body transform relating the two contacting monomers, but also those generated by the n-th root of this transform, where n ranges from 2 to 5—with a choice of n=4, for example, the 1st monomer will be in contact with the 4th monomer (FIG. 1C, bottom, FIG. 5). We also considered filaments with cyclic symmetry generated by application of Cn symmetry operations around the superhelical axis, where n is between 2 and 5 (FIG. 1C, middle). In all cases, we then generated several repeating turns of the full filament by repeated application of the rigid body transformation and cyclic symmetry operations, eliminate geometries with clashing subunits, and require the existence of at least one additional interface beyond that generated in the initial sliding into contact step. Filament architectures with multiple interacting surfaces predicted to have low energy after design were selected, and combinatorial sequence optimization was carried out on a central monomer, propagating the sequence to all other monomers. The resulting designs, which span the range of helical parameters (diameter, rise, and rotation, table 1) of native filaments (FIG. 1B), were filtered for high shape complementarity, low monomer-monomer interaction energy and few or no buried unsatisfied hydrogen bonds.

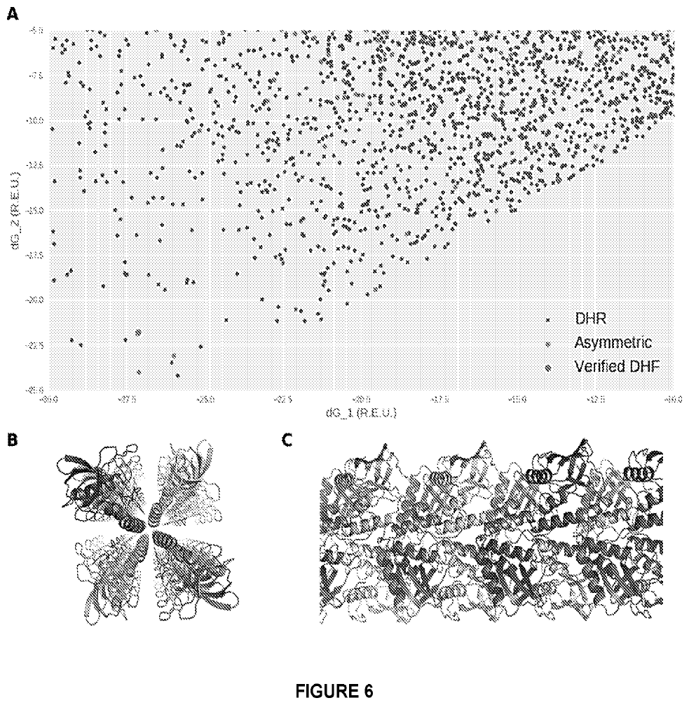

We chose as the monomeric building blocks a set of 15 de novo Designed Helical Repeat proteins (DHRs) which span a wide range of geometries and hence can give rise to a wide range of filament architectures. In addition to shape diversity, the DHRs have the advantages of very high stability and solubility, and are likely to tolerate the substitutions needed to design the multiple interfaces required to drive filament formation. They can also be extended or shortened simply by addition or removal of one or more of the 30-60 residue repeat units, potentially allowing tuning of the diameter of designed filaments. Starting from both the computational design models and the x-ray crystal structures of the DHRs, we generated 230000 helical filament backbones as described above and selected 124 designs for experimental testing (we refer to these as de novo Designed Helical Filaments or DHFs throughout the text; for comparison with filaments generated from native backbones, see FIG. 6).

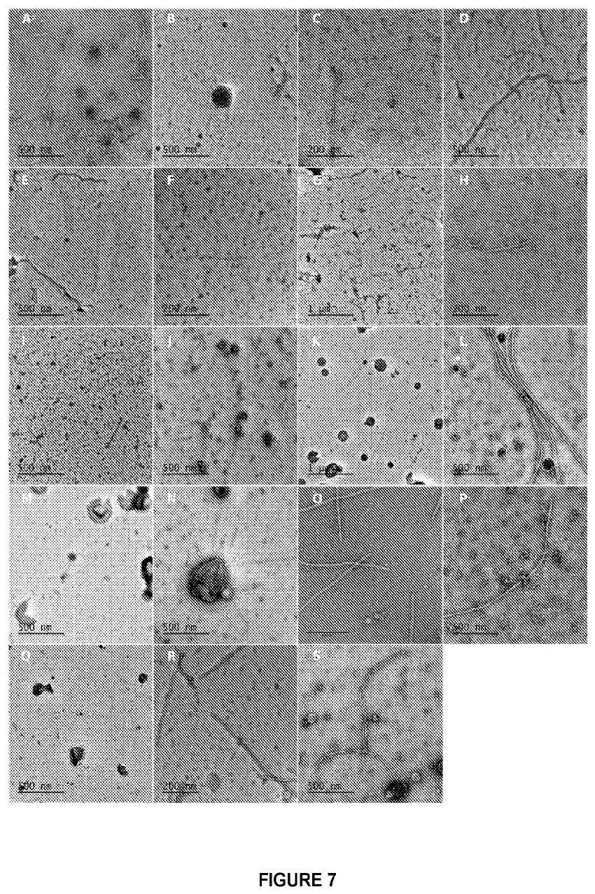

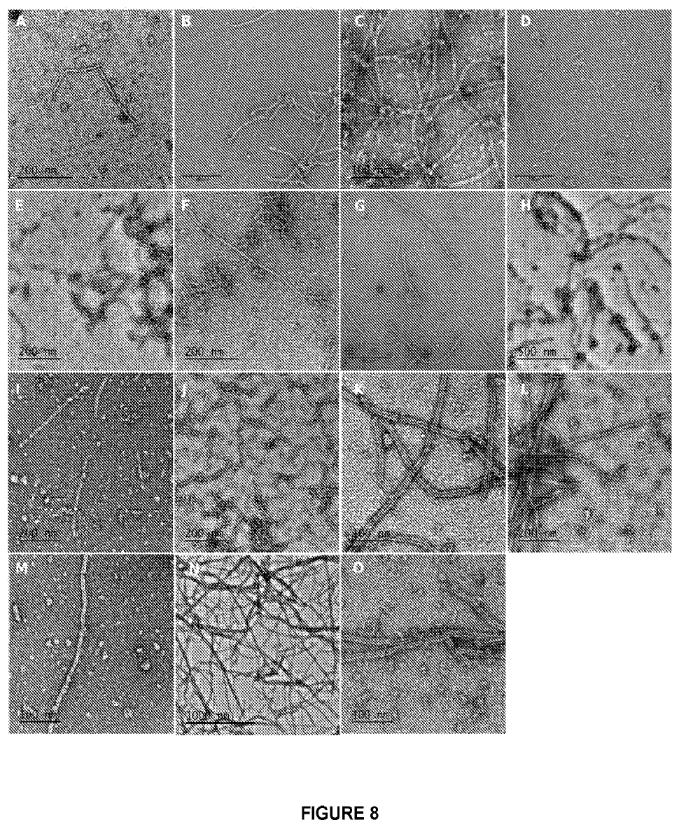

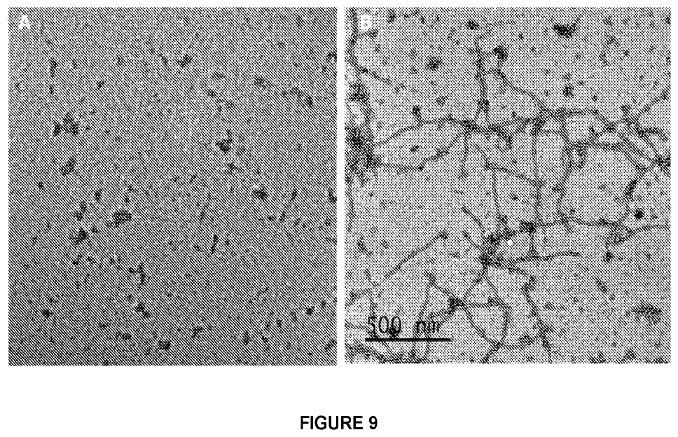

The designs were expressed in Escherichia coli under the control of a T7 promoter and purified using immobilized metal affinity chromatography (IMAC). Eighty-five of the designs were recovered in the IMAC eluate, while 22 were in the insoluble fraction (17 designs were not found in either fraction). IMAC eluates were concentrated, and filament formation was monitored by negative stain electron microscopy (EM); insoluble designs were characterized by EM either directly in the initial insoluble fraction, or after solubilization in guanidine hydrochloride, IMAC, and subsequent removal of denaturant. A total of 34 designs (15 soluble and 19 insoluble) were found to form one-dimensional nanostructures (FIGS. 7 and 8; the sequences are provided in the disclosure). A subset of the designs was synthesized as SUMO™ fusions to prevent premature filament formation; the SUMO™ tag was removed using SUMO™ protease and the samples characterized by negative stain EM (FIG. 9).

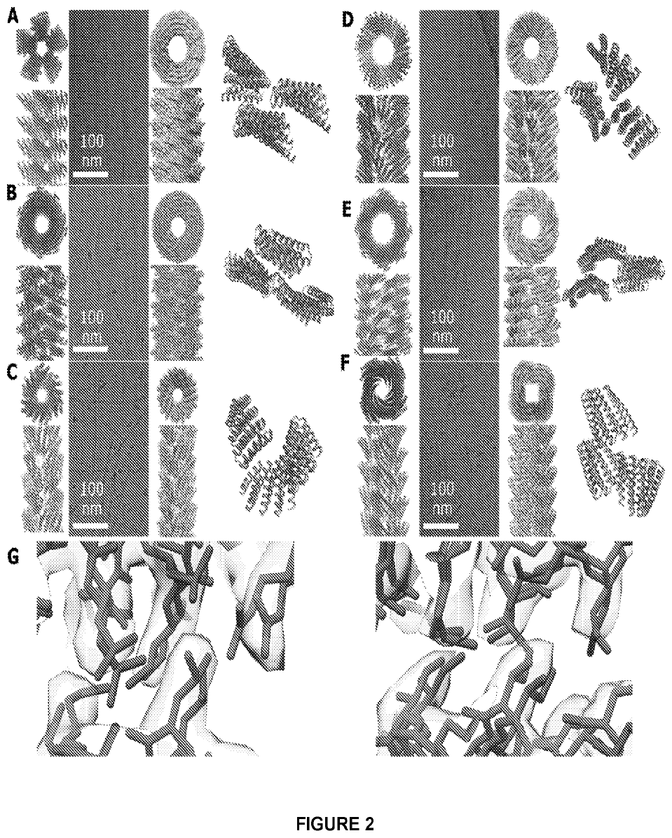

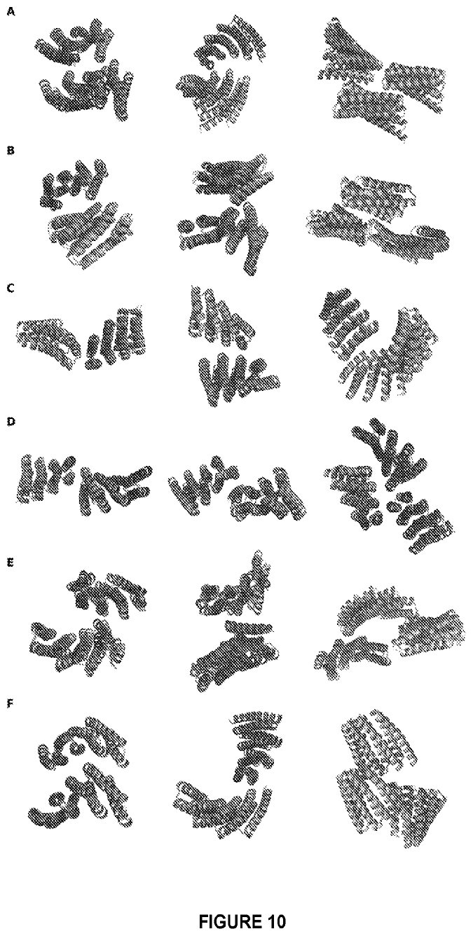

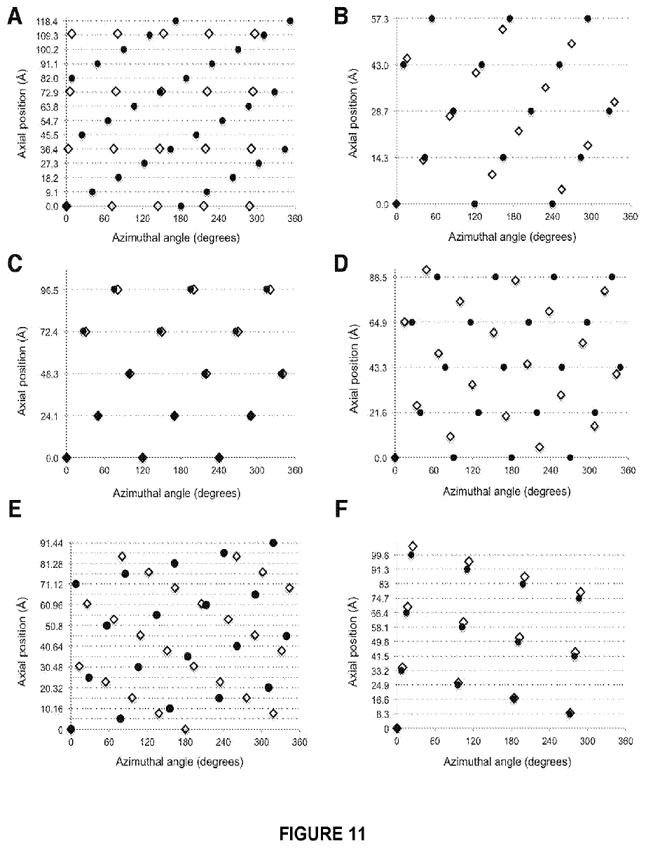

We chose six designs with a range of model architectures and highly ordered negative stain EM morphologies for higher resolution structure determination by cryo-electron microscopy (cryoEM). We determined the filament structures and refined helical symmetry parameters using iterative helical real space reconstruction in SPIDER™ (21, 22), followed by further 3D refinement in Relion™ (23) and Frealign™ (24). In all six cases, the overall orientation and packing of the monomers in the filament were similar in the experimentally determined structures and design models, but there was considerable variation in the accuracy with which the details of the interacting interfaces were modeled (FIG. 2, FIG. 10). Subtle shifts in the interaction interfaces in several cases altered the designed symmetry; DHF119 for example, was designed to be Cl but the cryoEM structure has C3 symmetry (helical lattice plot comparisons are in FIG. 11). Four of the six designed filaments matched the computational models at near-atomic resolution: for DHF38 and DHF 91 the experimentally observed rigid body orientation was nearly identical to the design models (0.9 Å and 1.2 Å r.m.s.d. over three chains containing all unique interfaces), for DHF46 and DHF119 the r.m.s.d. over three chains was 2.3 Å, and for DHF91 and DHF58, 3.6 and 4 Å. The structure of DHF119 was solved to 3.4 Å resolution; the backbone and side chain conformations at the subunit interfaces are very similar to those in the design model (FIG. 2G).

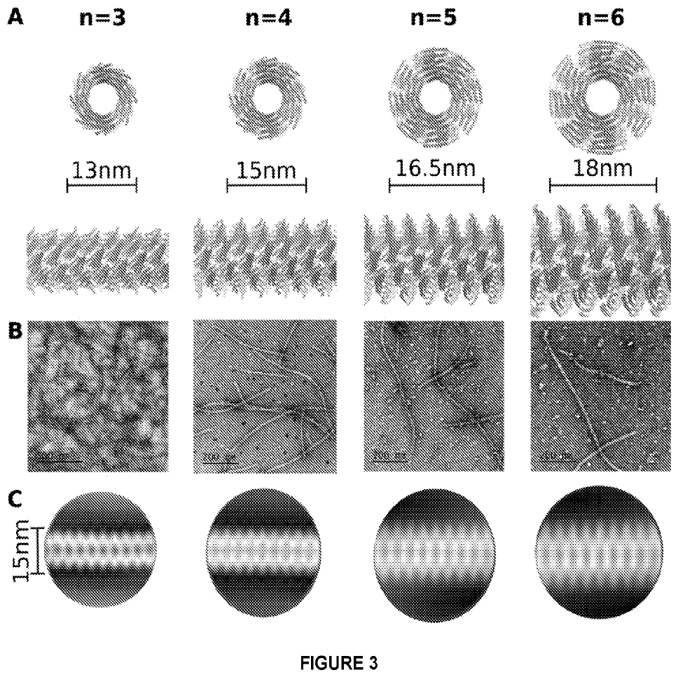

To determine whether the filament diameter could be modulated by changing the number of repeat units in the monomer, we generated a series of DHF58 variants that retain the fiber interaction interfaces but have three, four, five or six repeats in the protomer. The designs were expressed, purified and characterized by negative stain EM: consistent with the computational models (FIG. 3A), the diameter of the filaments changes linearly with the number of repeat units (FIG. 3B, C).

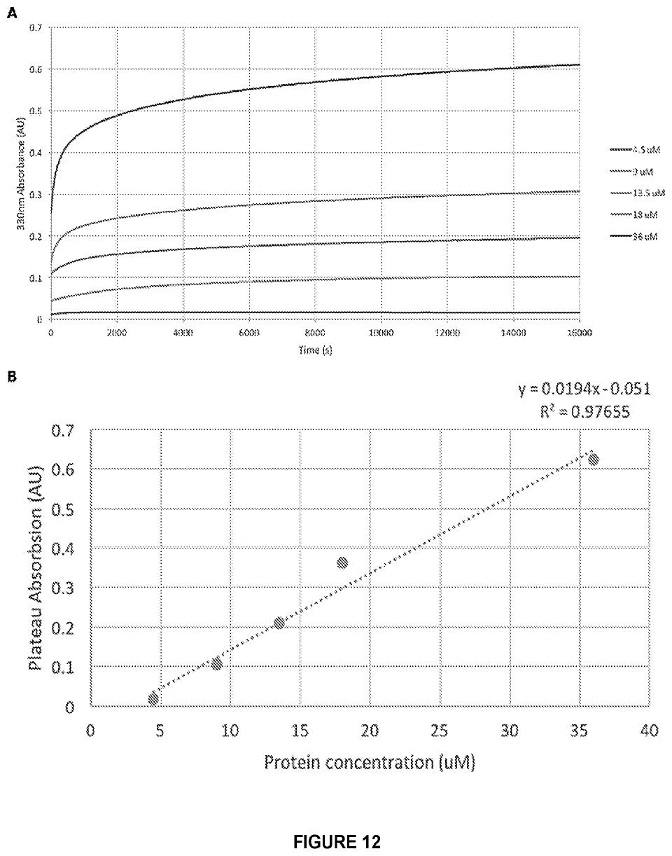

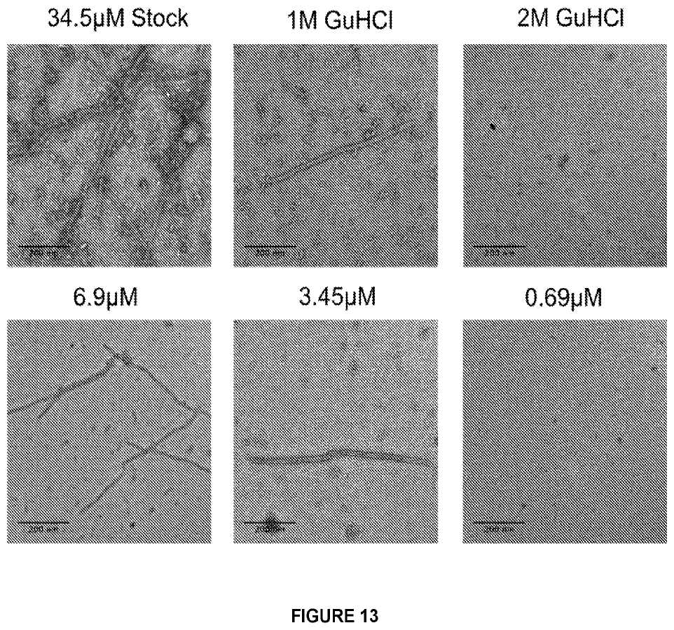

We monitored assembly dynamics in vitro by solution scattering and in living cells using fluorescence microscopy with monomers fused to green fluorescent protein (GFP). The extent and kinetics of DHF119 filament formation in vitro was strongly concentration-dependent. Filament nucleation was too fast to observe by manual mixing; the rate of the observed elongation phase was linear with respect to monomer concentration, and extrapolation of the plateau values from progress curves back to zero yielded a critical concentration of 3 (FIG. 12). Upon dilution below the critical concentration, filaments disassembled in several hours (FIG. 13). In E. coli following induction of expression of DHF58-GFP, filaments up to microns in length were observed (data not shown).

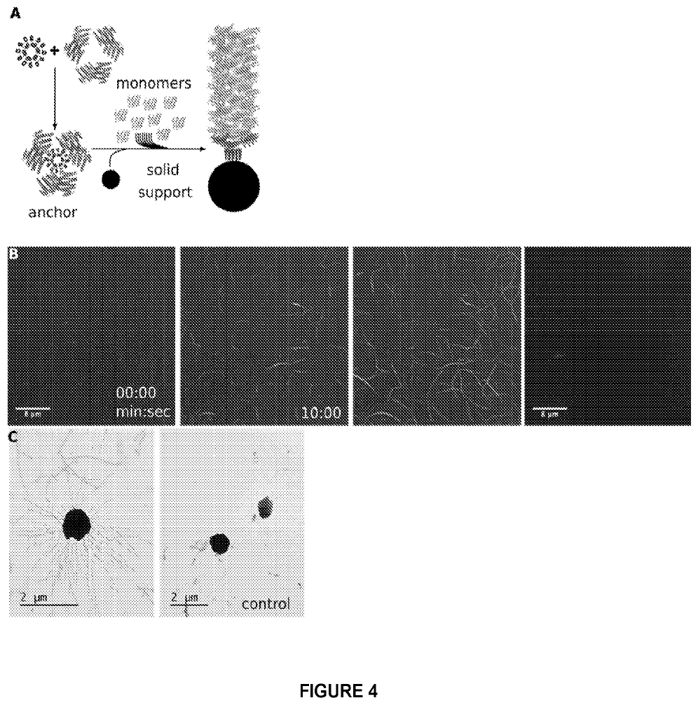

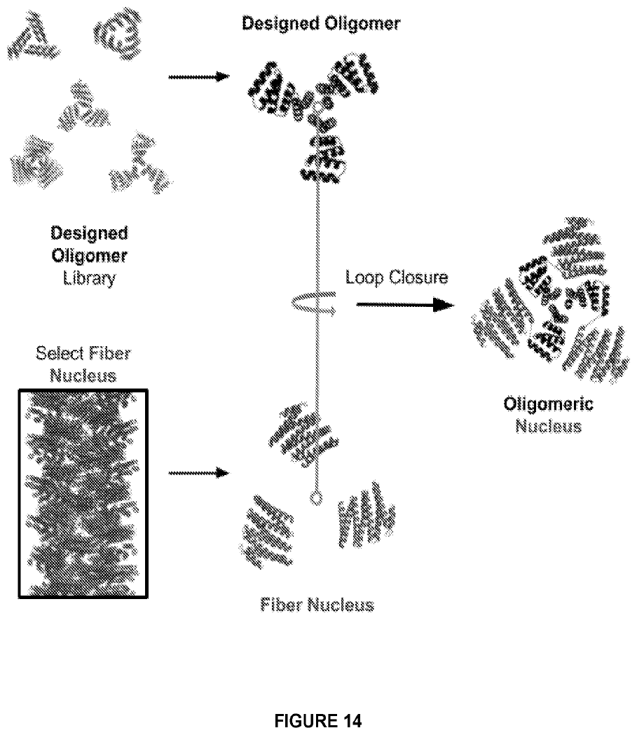

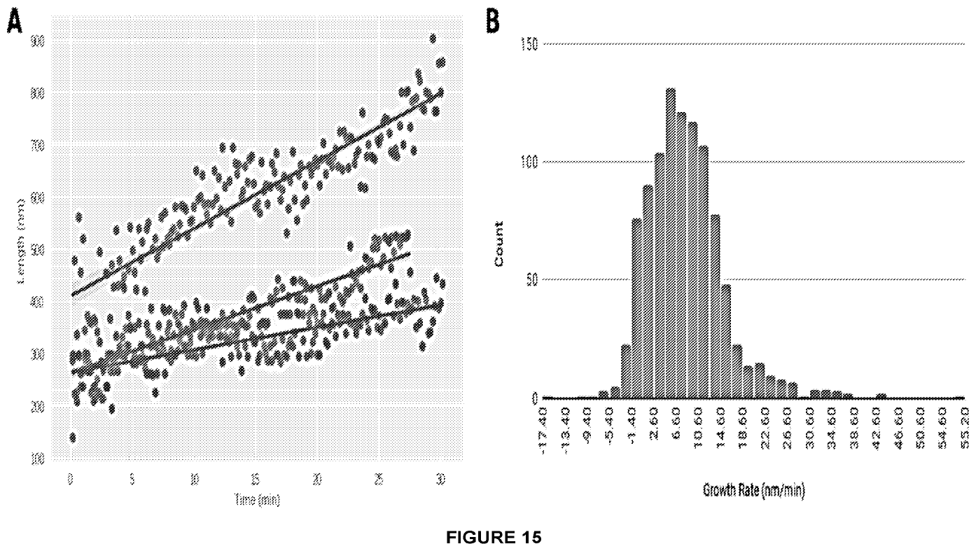

Natural systems achieve remarkable complexity and diversity of filament-based structures through modulating the nucleation, growth, and cellular location of the polymers. In some natural systems, nucleation and location are controlled by complexes that act as templates that initiate new growth and anchor filaments to specific locations, like the gamma-tubulin ring complex for microtubules and the Arp2/3 complex for actin. We sought to replicate this mechanism of control by designing multimeric anchor constructs, with multiple monomeric subunits held close to the relative orientations in the corresponding filaments by a fusion to designed homo-oligomers with the appropriate geometry (FIG. 14; one of the interaction interfaces is eliminated to restrict fiber growth in one direction). For example, anchor DHF119_C6 (FIG. 4B) is a hexamer in which each monomer consists of a designed oligomerization domain fused to the fiber monomer; the orientations of the monomers in the hexamer are close to those in the filament structure to promote both nucleation and fiber attachment. To study the kinetics of filament formation in vitro in more detail, we attached the anchors to glass slides, added monomers fused to yellow fluorescent protein (YFP) and monitored fiber formation by total internal reflection (TIRF) microscopy. The anchors seeded the rapid growth of multiple micron length fibers over 30 minutes (FIG. 4C; for analysis of growth kinetics of a second fiber see FIG. 15). Few or no fibers were observed to grow from the glass slide surface when it was coated with an anchor designed for a different fiber, or with no anchor at all. Attachment of biotinylated anchor to streptavidin-coated beads, followed by incubation with filament monomer resulted in an extensive network of filaments emanating from the beads (FIG. 4D, left panel); in contrast, very few filaments were observed around control beads that lacked the anchor protein (FIG. 4D, right panel).

To determine whether filament dissolution could also be modulated by designed accessory proteins, we produced monomeric capping units lacking one of the two designed interfaces in the DHF119 filament—these caps are expected to add to one end of the filament, but not the other, preventing further elongation (since the two ends of the filaments are distinct, there are two types of caps). Addition of increasing concentrations of the caps to already formed filaments resulted in shrinking and ultimately disappearance of the filaments (data not shown), suggesting that filaments are dynamically exchanging protomers at equilibrium. In the absence of caps, increasing the monomer concentration led to growth of the fibers at a rate observed by fluorescence for anchored fiber growth (8.4 nm/minute at 18 μM monomer, FIG. 15). The observed behavior can be understood as follows: at the critical monomer concentration where fibers neither grow or shrink, the (concentration dependent) rate of monomer addition to the ends is balanced by the (concentration independent) disassociation rate. Caps perturb this balance by complexing with monomers effectively reducing the free monomer concentration, hence when both end caps are present, disassembly wins out over growth, leading to a net shrinking of the filaments.

The ability to program micron scale order from Angstrom scale designed interactions between asymmetric monomers is an advance for computational protein design. In contrast to previous nanomaterial design efforts relying on an already existing interface within symmetric building blocks, proper assembly includes the design of two independent interfaces. The filaments described here are built from monomeric building blocks and have a wide range of geometries since only a small fraction of possible helical assemblies contain dihedral point group symmetry. Both designed interfaces were accurately recapitulated in four of the six structures solved by cryoEM; despite the deviations in the interfaces in the other two, the overall filament architecture was reasonably well recapitulated. The ability to program filament dynamics provides a baseline for understanding the much more complex regulation of the dynamic behavior of naturally occurring filaments. The repeat protein building blocks are hyperstable proteins robust to genetic fusion, and hence the designed filaments provide readily modifiable scaffolds to which binding sites for other proteins or metal nanoclusters can be added for applications ranging from cryoEM structure determination to nano-electronics.

### REFERENCES

1. S. Ricard-Blum, F. Ruggiero, M. van der Rest, in Collagen, J. Brinckmann, H. Notbohm, P. K. Müller, Eds. (Springer Berlin Heidelberg, Berlin, Heidelberg, 2005), vol. 247 of Topics in Current Chemistry, pp. 35-84.
2. L. C. Serpell, Alzheimer's amyloid fibrils: structure and assembly. Biochim. Biophys. Acta. 1502, 16-30 (2000).
3. H. Hellmann, U. Aebi, Intermediate filaments: molecular structure, assembly mechanism, and integration into functionally distinct intracellular Scaffolds. Annu. Rev. Biochem. 73, 749-789 (2004).
4. G. J. Rucklidge, G. Milne, B. A. McGaw, E. Milne, S. P. Robins, Turnover rates of different collagen types measured by isotope ratio mass spectrometry. Biochim. Biophys. Acta. 1156, 57-61 (1992).
5. K. C. Holmes, D. Popp, W. Gebhard, W. Kabsch, Atomic model of the actin filament. Nature. 347, 44-49 (1990).
6. E. Nogales, M. Whittaker, R. A. Milligan, K. H. Downing, High-Resolution Model of the Microtubule. Cell. 96, 79-88 (1999).
7. B. Bhyravbhatla, S. J. Watowich, D. L. D. Caspar, Refined Atomic Model of the Four-Layer Aggregate of the Tobacco Mosaic Virus Coat Protein at 2.4-Å Resolution. Biophys. J. 74, 604-615 (1998).
8. A. M. Smith et al., Polar assembly in a designed protein fiber. Angew. Chem. Int. Ed Engl. 44, 325-328 (2004).
9. L. E. R. O'Leary, J. A. Fallas, E. L. Bakota, M. K. Kang, J. D. Hartgerink, Multi-hierarchical self-assembly of a collagen mimetic peptide from triple helix to nanofibre and hydrogel. Nat. Chem. 3, 821-828 (2011).
10. C. J. Bowerman, B. L. Nilsson, Self-assembly of amphipathic (3-sheet peptides: insights and applications. Biopolymers. 98, 169-184 (2012).
11. J. D. Hartgerink, J. R. Granja, R. A. Milligan, M. Reza Ghadiri, Self-Assembling Peptide Nanotubes. J. Am. Chem. Soc. 118, 43-50 (1996).
12. E. H. Egelman et al., Structural plasticity of helical nanotubes based on coiled-coil assemblies. Structure. 23, 280-289 (2015).
13. N. C. Burgess et al., Modular Design of Self-Assembling Peptide-Based Nanotubes. J. Am. Chem. Soc. 137, 10554-10562 (2015).
14. C. Xu et al., Rational design of helical nanotubes from self-assembly of coiled-coil lock washers. J. Am. Chem. Soc. 135, 15565-15578 (2013).
15. F. A. Tezcan, F. Akif Tezcan, in Coordination Chemistry in Protein Cages (2013), pp. 149-174.
16. Y. Hsia et al., Corrigendum: Design of a hyperstable 60-subunit protein icosahedron. Nature. 540, 150 (2016).
17. N. P. King et al., Accurate design of co-assembling multi-component protein nanomaterials. Nature. 510, 103-108 (2014).
18. S. Gonen, F. DiMaio, T. Gonen, D. Baker, Design of ordered two-dimensional arrays mediated by noncovalent protein-protein interfaces. Science. 348, 1365-1368 (2015).
19. J. A. Fallas et al., Computational design of self-assembling cyclic protein homo-oligomers. Nat. Chem. 9, 353-360 (2017).
20. T. J. Brunette et al., Exploring the repeat protein universe through computational protein design. Nature. 528, 580-584 (2015).
21. E. H. Egelman, The iterative helical real space reconstruction method:

surmounting the problems posed by real polymers. J. Struct. Biol. 157, 83-94 (2007).
22. E. H. Egelman, A robust algorithm for the reconstruction of helical filaments using single-particle methods. Ultramicroscopy. 85, 225-234 (2000).
23. S. H. W. Scheres, RELION: implementation of a Bayesian approach to cryoEM structure determination. J. Struct. Biol. 180, 519-530 (2012).
24. N. Grigorieff, FREALIGN: high-resolution refinement of single particle structures. J. Struct. Biol. 157, 117-125 (2007).
25. H. Garcia-Seisdedos, C. Empereur-Mot, N. Elad, E. D. Levy, Proteins evolve on the edge of supramolecular self-assembly. Nature. 548, 244-247 (2017).
26. G. Bhardwaj et al., Accurate de novo design of hyperstable constrained peptides. Nature. 538, 329-335 (2016).
27. F. W. Studier, Protein production by auto-induction in high density shaking cultures. Protein Expr. Purif 41, 207-234 (2005).
28. B. L. Nannenga, M. G. Iadanza, B. S. Vollmar, T. Gonen, Overview of Electron Crystallography of Membrane Proteins: Crystallization and Screening Strategies Using Negative Stain Electron Microscopy. Curr. Protoc. Protein Sci. 72, 17.15.1-17.15.11 (2013).
29. J. Schindelin et al., Fiji: an open-source platform for biological-image analysis. Nat. Methods. 9, 676-682 (2012).
30. C. Suloway et al., Automated molecular microscopy: the new Leginon system. J. Struct. Biol. 151, 41-60 (2005).
31. S. Q. Zheng et al., MotionCor2: anisotropic correction of beam-induced motion for improved cryo-electron microscopy. Nat. Methods. 14, 331-332 (2017).
32. K. Zhang, Gctf: Real-time CTF determination and correction. J. Struct. Biol. 193, 1-12 (2016).
33. G. C. Lander et al., Appion: an integrated, database-driven pipeline to facilitate EM image processing. J. Struct. Biol. 166, 95-102 (2009).
34. C. Sachse et al., High-resolution Electron Microscopy of Helical Specimens: A Fresh Look at Tobacco Mosaic Virus. J. Mol. Biol. 371, 812-835 (2007).
35. P. D. Adams et al., in International Tables for Crystallography (2012), pp. 539-547.
36. P. Emsley, B. Lohkamp, W. G. Scott, K. Cowtan, Features and development of Coot. Acta Crystallogr. D Biol. Crystallogr. 66, 486-501 (2010).
37. A. E. Carpenter et al., CellProfiler: image analysis software for identifying and quantifying cell phenotypes. Genome Biol. 7, R100 (2006).

### Materials and Methods

Generation of Filament Models from Monomeric Building Blocks. The goal of the computational helix docking procedure was to exhaustively sample, to within some specified resolution and acceptable interface quality, all possible ways to build a symmetric helix from a monomeric building block. We start by enumerating all possible head-to-tail dimeric arrangements of the monomer. The six-dimensional rigid body docking space of three rotations and three translations is reduced to five by requiring contact between the bodies; the three translational degrees of freedom are replaced by a two-dimensional space of normal vector directions and a slide into contact (FIG. 1C, left). Experience suggests this reduction works reasonably well for globular bodies (19). The resulting dimer interface is scored using the RPX method, and those below a cutoff are discarded. Given a head-to-tail homodimeric interface X, docking proceeds by generating possible helix geometries containing interface X along with at least one other interface Y. Two discrete parameters N and C determine what helices can be constructed by repeating interface X. Parameter C specifies the cyclic symmetry of the result (FIG. 1C middle), and parameter N specifies how many helix unit transforms are needed to produce interface X (FIG. 1C bottom). Given X, N, and C, a rapid check of the helical spacing is performed, as most combinations will result in clashing or overly extended helices without a second protein interface. If this check passes, the geometry is explicitly generated and checked for the presence of a second homomeric interface Y. Interface Y is then scored using RPX, and the score for the overall helix is the worst of X and Y.

Interface Design. Docks with appropriate and evenly-distributed interface sizes as well as good RPX scores (19) were selected to perform interface sequence design in RosettaScripts™. In each design trajectory, the protomer was initially perturbed by a random rotation around its center of mass. A polymer with the specified helical symmetry was generated using the information stored in the symmetry definition file, which was generated from the initial docking configuration using tools distributed with the Rosetta™ Macromolecular Modeling suite. Amino acids at the interface were optimized using Monte Carlo simulated annealing protocol available in the Rosetta™ Macromolecular Modeling suite. An initial optimization step was executed with the rotamers available in the database of residue-pair motifs and a modified score function with a down-weighted repulsive term. Once a sequence was converged on, designable positions were allowed to minimize side-chain torsion angles. A subsequent round of minimization was conducted with the standard score function to obtain a conformation that corresponds to a local minimum of the energy function. Individual design trajectories were filtered by the following criteria: the difference between the Rosetta™ energy of the bound (polymeric) and unbound (monomeric) states less than −15.0 Rosetta™ Energy Units, interface surface area greater than 700 Å², Rosetta™ shape complementarity greater than 0.62 and unsatisfied polar residues less than 5. Designs that passed these criteria were manually inspected and refined by single-point reversions for mutations that were deemed not to contribute to stabilizing the bound state of the interface. The design with the best overall scores for each docked configuration was then added to a set of finalized proteins to be validated experimentally.

Accessory Protein Design. Capping units for DHF58 and DHF119 were designed by mutating the residue identities at the interfaces that drive filament growth to identities in the corresponding scaffold proteins. Capping proteins with reversions in primary sequence close to the N-terminus are referred to as N caps while proteins with reversions in the primary sequence at the C-terminal end are referred to as C-caps. The anchor protein DHF119_C6 was designed by fusing the monomer from designed hexamer 3H22 to the C cap of DHF 119 with a (GGS)5 linker. An avi-tag (GLNDIFEAQKIEWHE; SEQ ID NO:41) was added to the N terminus of 3H22 for biotinylation.

Protein Expression and Purification. Synthetic genes for 124 designs were optimized for E. coli expression and purchased from Gen9 and Genscript ligated in the multiple cloning site of the pET28b vector between Ndel and Xhol restriction sites or in vector pCDB24 (26). This vector contains SUMO protein Smt3 from Saccharomyces cerevisiae to prevent premature assembly in E. coli and improve solubility. These plasmids were cloned into BL21*(DE3) (Invitrogen) E. coli competent cells. Transformants were inoculated into 50 ml of TB medium with 200 mg L⁻¹ kanamycin. Expression proceeded for 24 hours at 37° C. following the expression via Studier autoinduction (27) until the cultures were harvested by centrifugation. Cell pellets were resuspended in TBS and lysed using the Bugbuster™ detergent (Millipore). The soluble fraction upon lysate clarification by centrifugation was purified by Ni²⁺ immobilized metal affinity chromatography with Ni-NTA Superflow resin (Qiagen). Resin with bound cell lysate was washed with 10 column volumes of 40 mM imidazole and 500 mM NaCl and eluted with 400 mM imidazole and 75 mM NaCl. Both the soluble and insoluble fractions were run on an SDS-PAGE gel. Samples that showed protein bands at the correct molecular weight were selected for screening by electron microscopy. Proteins expressed in the pCDB24 vector were screened before and after cleavage of the fusion protein using the SUMO™ protease (FIG. S5). Selected designs were expressed at the 0.5 L scale to carry out further characterization. Expression proceeded for 24 hours at 37° C. following the expression via Studier autoinduction (27) until the cultures were harvested by centrifugation. Cell pellets were resuspended in TBS and lysed by microfluidization. Purification was carried out as described above.

Negative Stain Electron Microscopy. Soluble fractions were concentrated and insoluble fractions were resuspended in buffer (25 mM Tris, 75 mM NaCl, pH 8) for EM screening. A drop of 6 μL (1 μl sample instantly diluted with 5 μl of buffer) was applied on negatively glow discharged, carbon-coated 200-mesh copper grids (Ted Pella, Inc.), washed with Milli-Q™ Water and stained using 0.75% uranyl formate as described previously (28). The screening was performed on either a 120 kV Tecnai Spirit™ T12 transmission electron microscope (FEI, Hillsboro, Oreg.) or a 100 kV Morgagni M268 transmission electron microscope (FEI, Hillsboro, Oreg.). Images were recorded on a bottom mount Teitz CMOS™ 4 k camera system. The contrast of the images was enhanced in the Fiji software (29) for clarity.

CryoEM Sample Preparation and Data Collection. CryoEM samples were prepared by applying protein to glow-discharged C-Flat holey-carbon grids (Protochips Inc.), blotting with a Vitrobot™ (FEI co.), and plunging into liquid ethane. For DHF58, DHF46, DHF79, and DHF91 samples, data was collected on a Tecnai G2 F20 (FEI co.) operating at 200 kV with a K-2 Summit Direct Detect camera (Gatan Inc.) with a pixel size of 1.26 Å/pixel. Movies were acquired in counting mode with 36 frames and a total dose of ˜45 e⁻/Å². For DHF119 and DHF38 samples, data was collected on a Titan Krios™ (FEI co.) operating at 300 kV, with a Quantum GIF energy filter (Gatan Inc.) operating in zero-loss mode with a 20 eV slit width, and a K-2 Summit™ Direct Detect camera with a pixel size of 0.525 Å/pixel. Movies were acquired in super-resolution mode with 50 frames and a total dose of ˜90 e⁻/Å². All data was collected with a defocus range between 1.0 and 2.5 μm, using Leginon™ (30) or EPU™ (FEI co.) software for automated data collection.

Image Processing, 3D Reconstruction, and Model Building. Movie frames were aligned and dose-weighted using MotionCor2™ (31) and CTF values were determined using GCTF (32). Helices were picked manually using Appion™ (33) or Relion™ (23) software, and particles were extracted as overlapping segments along the length of each helix. Reference-free 2D classification of helical segments was then performed using Relion™. For DHF119 and DHF38, selected 2D classes obtained from manual picking of a subset of images were used as templates for automated picking in Relion™. Following 2D classification of all particles, particles from good classes were selected for subsequent 3D reconstructions. Initial 3D reconstructions were performed by iterative helical real space reconstruction (IHRSR) (21, 34) in SPIDER™, using cylinders as starting models, and using hsearch_lorentz (22) to refine helical symmetry parameters. In cases where additional point group symmetry became apparent, this was enforced in subsequent rounds of refinement. Gold-standard refinement in SPIDER™ was performed with increasingly smaller angular sampling, with a minimum sampling of 1.5°. For DHF119, DHF38, DHF79, and DHF91, further 3D helical refinement was performed using Relion™, using the values determined by hsearch_lorentz as initial helical symmetry parameters, and the SPIDER™ volumes (low-pass filtered to 30 Å) as starting models. For DHF38, angles and shifts determined by Relion were further refined by local refinement in Frealign™ MODE 1 (24). For DHF58 and DHF46, volumes were amplitude corrected and low/high-pass filtered in SPIDER™. For DHF119, DHF38, DHF79, and DHF91, volumes were B-factor sharpened and low-pass filtered using Relion post-processing. The gold-standard FSC=0.143 criterion was used for estimating resolution. Atomic models were fit into cryoEM density as rigid bodies. For DHF119 and DHF38, atomic models were further refined by real-space refinement in Phenix (35) and Coot (36).

Filament Growth In Vitro. PEG-silane coated glass coverslips were attached to similarly-coated slides with strips of double-stick tape to make flow chambers. All incubations were at 25° C. Dry glass chambers were coated for 2 minutes with 8 mg/ml kappa-casein (Sigma C0406) 10:1 biotinylated casein in BRB80 (80 mM PIPES-KOH ph 6.85+1 mM MgCl₂+1 mM EGTA), washed twice with CK buffer (BRB80+1 mg/ml casein+70 mM KCl), incubated 3 minutes with 0.5 mg/ml neutravidin (Molecular Probes A2666) in CK, then washed three times with CK. Prepared cells were washed once in IB (imaging buffer: 75 mM NaCl+25 mM Tris-HCl pH 8.0+11 mM glucose+2.5 mM DTT+0.2 mg/ml glucose oxidase (Sigma G2133)+0.04 mg/ml catalase (Sigma C40). Biotinylated anchor protein (DHF119_C6 with C-terminal GFP fusion) 36.6 nM in IB was incubated in chamber for 3 minutes, chamber washed twice with IB, and replaced with 1.16 μM DHF119-YFP in IB for observation of assembly. Imaging was carried out using a Personal Deltavision™ microscope (GE Healthcare) outfitted with 4-laser TIRF capabilities, Olympus 60x, 1.49 NA TIRF objective and Ultimate focus (Applied Precision) at room temperature.

For analysis of growth kinetics of DHF119-GFP fiber: Images were processed for subsequent analysis in CellProfiler™. The background of the tif movies was subtracted using the software Fiji (29) with a ball radius of 5.0. The contrast was modified to reduce background noise to facilitate fiber identification. CellProfiler™ software (37) was used to identify and track fiber through the different time frames. The output files from CellProfiler™ provided the Major axis length for every fiber through all the movie.

TABLE 1
Computational and experimental parameters for all designs tested.

| Computation | | | | | | | | | | | |
|---|---|---|---|---|---|---|---|---|---|---|---|
| | | | | | | | dG_1 | | | | |
| | | | Rotation | Radius | | | (R.E. | dG_2 | Experiment | | |
| Design | Cn | Rise(Å) | (°) | (Å) | SC_1^{¶} | SC_2^{§} | U.)^{†} | (R.E.U.)^{‡} | vector | fiber* | solubility^{#} |
| DHF1 | 1 | 7.5 | −82.7 | 23.5 | 0.9 | 0.7 | −19.2 | −10.0 | pET28b | 0 | 2 |
| DHF2 | 1 | 7.8 | 130.6 | 27.0 | 0.7 | 0.7 | −23.6 | −19.3 | pET28b | 0 | 0 |
| DHF3 | 1 | 9.4 | 131.1 | 28.6 | 0.9 | 0.7 | −19.5 | −10.5 | pET28b | 0 | 2 |
| DHF4 | 1 | 7.7 | −86 | 28.2 | 0.8 | 0.8 | −25.6 | −20.9 | pCDB24 | 1 | 1 |
| DHF5 | 1 | 9.0 | 131.9 | 27.0 | 0.7 | 0.7 | −25.0 | −22.5 | pCDB24 | 1 | 1 |
| DHF6 | 1 | 11.7 | −151.4 | 24.3 | 0.8 | 0.7 | −15.7 | −7.3 | pCDB24 | 0 | 2 |
| DHF7 | 1 | 6.7 | 82.9 | 31.5 | 0.8 | 0.7 | −18.9 | −8.9 | pCDB24 | 0 | 2 |
| DHF8 | 1 | 14.6 | 94.1 | 19.8 | 0.7 | 0.6 | −22.7 | −1.6 | pET28b | 1 | 1 |
| DHF9 | 1 | 12.1 | −152 | 24.5 | 0.8 | 0.8 | −8.7 | −7.8 | pET28b | 1 | 2 |
| DI1F10 | 1 | 9.3 | 132.9 | 27.8 | 0.8 | 0.7 | −18.7 | −12.9 | pET28b | 0 | 2 |
| DHF11 | 2 | 4.2 | −31.5 | 74.1 | 0.6 | 0.6 | −32.3 | −30.5 | pCDB24 | 0 | 0 |
| DHF12 | 1 | 7.8 | 130.2 | 26.6 | 0.8 | 0.7 | −17.7 | −14.9 | pCDB24 | 0 | 2 |
| DHF13 | 1 | 11.8 | −150.3 | 24.4 | 0.7 | 0.7 | −16.8 | −9.4 | pCDB24 | 0 | 2 |
| DHF14 | 1 | 11.5 | 84 | 26.3 | 0.7 | 0.7 | −21.4 | −14.7 | pCDB24 | 0 | 2 |
| DHF15 | 2 | 8.9 | 52.2 | 40.4 | 0.7 | 0.7 | −24.8 | −9.9 | pET28b | 0 | 0 |
| DHF16 | 1 | 8.7 | −109.1 | 28.8 | 0.8 | 0.6 | −6.3 | −5.6 | pET28b | 1 | 1 |
| DHF17 | 1 | 4.1 | 102.3 | 47.3 | 0.7 | 0.6 | −29.8 | −21.1 | pET28b | 1 | 1 |
| DHF18 | 2 | 10.2 | −79.4 | 35.1 | 0.8 | 0.7 | −10.9 | −1.0 | pCDB24 | 0 | 2 |
| DHF19 | 1 | 7.9 | 130.6 | 26.9 | 0.7 | 0.7 | −24.1 | −16.3 | pCDB24 | 0 | 2 |
| DHF20 | 1 | 4.8 | −139.9 | 44.1 | 0.7 | 0.7 | −20.5 | −13.9 | pCDB24 | 1 | 2 |
| DHF21 | 2 | 21.8 | −102.9 | 22.6 | 0.7 | 0.6 | −17.9 | −12.6 | pCDB24 | 0 | 2 |
| DHF22 | 2 | 14.6 | −71 | 30.2 | 0.7 | 0.7 | −18.6 | −15.2 | pET28b | 0 | 0 |
| DHF23 | 1 | 6.5 | 83 | 33.1 | 0.8 | 0.8 | −14.1 | −12.8 | pET28b | 1 | 1 |
| DHF24 | 1 | 3.4 | 48.8 | 63.4 | 0.9 | 0.7 | −17.7 | −10.6 | pCDB24 | 0 | 2 |
| DHF25 | 4 | 29.4 | 23.9 | 37.2 | 0.7 | 0.7 | −19.2 | −14.7 | pCDB24 | 1 | 1 |
| DHF26 | 1 | 7.9 | 130.1 | 26.7 | 0.9 | 0.7 | −22.8 | −15.0 | pCDB24 | 0 | 1 |
| DHF27 | 2 | 11.5 | −43.2 | 46.8 | 0.7 | 0.7 | −21.6 | −18.3 | pCDB24 | 0 | 2 |
| DHF28 | 1 | 10.4 | −108.6 | 25.5 | 0.7 | 0.6 | −16.3 | −7.8 | pCDB24 | 1 | 1 |
| DHF29 | 2 | 15.1 | −76.3 | 33.3 | 0.8 | 0.7 | −2.7 | −2.3 | pET28b | 0 | 2 |
| DHF30 | 1 | 8.0 | 131.9 | 27.0 | 0.8 | 0.8 | −27.1 | −11.9 | pET28b | 0 | 2 |
| DHF31 | 1 | 14.2 | 99 | 19.8 | 0.7 | 0.5 | −13.9 | −2.7 | pCDB24 | 1 | 1 |
| DHF32 | 4 | 22.7 | 25.7 | 38.3 | 0.8 | 0.7 | −12.1 | −8.0 | pCDB24 | 0 | 2 |
| DHF33 | 3 | 19.9 | 28.6 | 30.7 | 0.7 | 0.7 | −43.5 | −8.7 | pCDB24 | 0 | 2 |
| DHF34 | 1 | 8.4 | −110.3 | 27.2 | 0.8 | 0.7 | −22.4 | −19.7 | pCDB24 | 1 | 1 |
| DHF35 | 1 | 10.3 | −108.9 | 25.2 | 0.7 | 0.7 | −19.2 | −16.9 | pCDB24 | 0 | 1 |
| DHF36 | 1 | 5.9 | −147.2 | 33.7 | 0.8 | 0.7 | −15.5 | −6.3 | pET28b | 1 | 1 |
| DHF37 | 2 | 14.8 | −77.5 | 31.1 | 0.8 | 0.7 | −18.2 | −17.7 | pET28b | 0 | 1 |
| DHF38 | 1 | 8.7 | −87.9 | 30.0 | 0.8 | 0.7 | −15.7 | −9.7 | pCDB24 | 1 | 2 |
| DHF39 | 5 | 19.9 | 43.8 | 53.3 | 0.8 | 0.7 | −18.2 | −11.2 | pCDB24 | 0 | 2 |
| DHF40 | 1 | 5.4 | 139.9 | 37.6 | 0.7 | 0.7 | −29.5 | −6.6 | pCDB24 | 1 | 1 |
| DHF41 | 1 | 10.4 | −107.9 | 25.9 | 0.7 | 0.7 | −11.9 | −9.1 | pCDB24 | 0 | 2 |
| DHF42 | 1 | 7.1 | −135.6 | 29.6 | 0.8 | 0.8 | −17.1 | 0.2 | pET28b | 0 | 0 |
| DHF43 | 2 | 28.3 | 81.5 | 18.0 | 0.8 | 0.7 | −17.2 | −1.3 | pET28b | 1 | 1 |
| DHF44 | 1 | 14.5 | 97.4 | 19.7 | 0.7 | 0.5 | −23.6 | −4.6 | pCDB24 | 1 | 1 |
| DHF45 | 1 | 8.4 | −136 | 26.6 | 0.7 | 0.7 | −13.7 | −6.7 | pCDB24 | 0 | 2 |
| DHF46 | 1 | 5.0 | −137.3 | 36.5 | 0.7 | 0.7 | −13.7 | −13.3 | pET28b | 1 | 1 |
| DHF47 | 1 | 10.3 | −109.1 | 25.4 | 0.8 | 0.8 | −14.5 | −12.2 | pCDB24 | 1 | 1 |
| DHF48 | 1 | 10.1 | 135.2 | 26.9 | 0.8 | 0.7 | −18.6 | −8.3 | pET28b | 1 | 2 |
| DHF49 | 2 | 29.8 | −99.2 | 18.7 | 0.7 | 0.5 | −2.5 | 28.3 | pET28b | 1 | 1 |
| DHF50 | 1 | 14.5 | 94.9 | 19.8 | 0.7 | 0.6 | −24.7 | −4.2 | pCDB24 | 1 | 1 |
| DHF51 | 1 | 10.0 | 135 | 26.7 | 0.8 | 0.7 | −10.0 | −3.9 | pCDB24 | 1 | 2 |
| DHF52 | 1 | 5.2 | 148.7 | 31.1 | 0.7 | 0.7 | −9.0 | −3.7 | pCDB24 | 1 | 2 |
| DHF53 | 1 | 7.6 | −109.1 | 28.7 | 0.8 | 0.7 | −15.9 | −14.5 | pCDB24 | 0 | 2 |
| DHF54 | 1 | 14.4 | 94.6 | 19.9 | 0.8 | 0.7 | −25.1 | −2.4 | pET28b | 0 | 2 |
| DHF55 | 2 | 14.8 | −71 | 30.4 | 0.8 | 0.7 | −21.2 | −11.7 | pET28b | 0 | 0 |
| DHF56 | 2 | 22.4 | −108.8 | 22.4 | 0.7 | 0.6 | −22.6 | −16.9 | pET28b | 0 | 2 |
| DHF57 | 2 | 28.5 | 83 | 16.2 | 0.8 | 0.7 | −30.5 | −16.6 | pET28b | 0 | 2 |
| DHF58 | 5 | 36.7 | 3.1 | 33.6 | 0.8 | 0.7 | −17.4 | −5.1 | pET28b | 1 | 2 |
| DHF59 | 1 | 7.1 | −134.8 | 30.0 | 0.8 | 0.8 | −23.2 | −11.2 | pET28b | 0 | 2 |
| DHF60 | 1 | 14.0 | 98.7 | 22.8 | 0.7 | 0.7 | −29.6 | −4.3 | pET28b | 0 | 0 |
| DHF61 | 2 | 13.3 | −67.3 | 33.3 | 0.8 | 0.7 | −15.7 | −13.4 | pCDB24 | 0 | 2 |
| DHF62 | 2 | 28.0 | −100.8 | 23.2 | 0.8 | 0.7 | −16.0 | −4.9 | pET28b | 1 | 2 |
| DHF63 | 2 | 9.4 | 51.3 | 38.4 | 0.8 | 0.7 | −17.9 | −17.8 | pET28b | 0 | 0 |
| DHF64 | 3 | 27.8 | −95.1 | 27.4 | 0.8 | 0.7 | −11.2 | −9.4 | pET28b | 0 | 2 |
| DHF65 | 1 | 5.4 | −151.4 | 35.6 | 0.8 | 0.7 | −20.1 | −19.1 | pET28b | 0 | 2 |
| DHF66 | 1 | 13.4 | −134.1 | 20.3 | 0.8 | 0.7 | −14.6 | −2.2 | pET28b | 0 | 2 |
| DHF67 | 1 | 14.4 | −148.7 | 19.8 | 0.8 | 0.6 | −20.0 | −6.1 | pET28b | 0 | 2 |
| DHF68 | 1 | 13.2 | −151.7 | 23.2 | 0.8 | 0.7 | −19.0 | −8.6 | pET28b | 0 | 2 |
| DHF69 | 1 | 8.9 | −107.3 | 28.8 | 0.8 | 0.7 | −8.5 | −8.0 | pET28b | 0 | 2 |
| DHF70 | 1 | 6.8 | −85.2 | 32.8 | 0.8 | 0.7 | −14.6 | −13.7 | pET28b | 0 | 2 |
| DHF71 | 1 | 4.8 | 57.2 | 36.8 | 0.7 | 0.6 | −27.0 | −17.8 | pET28b | 0 | 0 |
| DHF72 | 1 | 9.2 | 88.6 | 27.1 | 0.7 | 0.7 | −19.8 | −11.1 | pET28b | 0 | 2 |
| DHF73 | 1 | 7.8 | −85.8 | 28.0 | 0.8 | 0.7 | −24.6 | −12.8 | pET28b | 0 | 2 |
| DHF74 | 2 | 17.2 | 60.8 | 26.8 | 0.7 | 0.7 | −15.5 | −0.7 | pET28b | 0 | 2 |
| DHF75 | 4 | 16.3 | 35.5 | 49.9 | 0.7 | 0.7 | −18.0 | −15.2 | pET28b | 0 | 2 |
| DHF76 | 1 | 5.3 | −151.9 | 35.5 | 0.8 | 0.7 | −19.6 | −6.1 | pET28b | 1 | 2 |
| DHF77 | 1 | 7.4 | 135.2 | 27.7 | 0.8 | 0.7 | −13.2 | −8.7 | pET28b | 1 | 1 |
| DHF78 | 1 | 12.6 | −147.5 | 24.6 | 0.8 | 0.7 | −24.5 | −17.8 | pET28b | 1 | 2 |
| DHF79 | 2 | 7.7 | −41.8 | 58.1 | 0.7 | 0.7 | −16.2 | −12.4 | pET28b | 1 | 2 |
| DHF80 | 1 | 4.1 | 79.1 | 60.7 | 0.8 | 0.7 | −15.0 | −9.7 | pET28b | 0 | 2 |
| DHF81 | 1 | 6.5 | −86.7 | 30.4 | 0.8 | 0.7 | −14.0 | −7.0 | pET28b | 0 | 2 |
| DHF82 | 1 | 4.5 | −84 | 46.9 | 0.8 | 0.7 | −21.2 | −0.2 | pET28b | 1 | 2 |
| DHF83 | 2 | 9.7 | 50.1 | 38.5 | 0.8 | 0.7 | −22.4 | −8.9 | pET28b | 0 | 0 |
| DHF84 | 1 | 5.9 | 68 | 34.1 | 0.8 | 0.7 | −19.4 | −10.2 | pET28b | 0 | 0 |
| DHF85 | 2 | 12.2 | 65.7 | 33.6 | 0.8 | 0.8 | −20.5 | −14.9 | pET28b | 0 | 0 |
| DHF86 | 2 | 13.2 | 70.8 | 30.1 | 0.7 | 0.7 | −11.8 | −11.3 | pET28b | 0 | 0 |
| DHF87 | 2 | 14.2 | −109.6 | 34.6 | 0.7 | 0.7 | −20.4 | −8.6 | pET28b | 0 | 2 |
| DHF88 | 2 | 7.0 | 31.5 | 65.1 | 0.8 | 0.7 | −14.9 | −13.0 | pET28b | 0 | 2 |
| DHF89 | 3 | 9.4 | 35.6 | 65.5 | 0.7 | 0.7 | −24.9 | −12.7 | pET28b | 0 | 2 |
| DHF90 | 3 | 29.0 | −85.9 | 24.3 | 0.7 | 0.7 | −13.1 | −12.8 | pET28b | 0 | 2 |
| DHF91 | 3 | 24.2 | 48.8 | 24.4 | 0.7 | 0.7 | −27.2 | −21.8 | pET28b | 1 | 2 |
| DHF92 | 3 | 22.4 | −84.6 | 32.0 | 0.8 | 0.7 | −11.6 | −7.9 | pET28b | 0 | 2 |
| DHF93 | 3 | 22.9 | −84.4 | 32.0 | 0.8 | 0.8 | −14.7 | −10.5 | pET28b | 0 | 2 |
| DHF94 | 3 | 21.7 | 40.8 | 31.8 | 0.7 | 0.7 | −16.8 | −14.1 | pET28b | 0 | 2 |
| DHF95 | 4 | 19.1 | 54.3 | 37.4 | 0.7 | 0.7 | −15.8 | −14.4 | pET28b | 0 | 2 |
| DHF96 | 4 | 37.1 | −4.8 | 26.8 | 0.8 | 0.8 | −22.7 | −10.8 | pET28b | 0 | 2 |
| DHF97 | 4 | 36.5 | 85.8 | 27.0 | 0.7 | 0.7 | −22.1 | −21.1 | pET28b | 0 | 2 |
| DHF98 | 4 | 36.7 | −4.1 | 27.1 | 0.7 | 0.7 | −22.8 | −16.7 | pET28b | 0 | 2 |
| DHF99 | 1 | 5.6 | −149.4 | 33.4 | 0.7 | 0.7 | −20.3 | −13.6 | pET28b | 0 | 0 |
| DHF100 | 1 | 4.1 | −156.8 | 40.9 | 0.8 | 0.6 | −17.1 | −10.8 | pET28b | 0 | 2 |
| DHF101 | 1 | 5.7 | −149.3 | 33.5 | 0.8 | 0.7 | −9.2 | −4.4 | pET28b | 0 | 2 |
| DHF102 | 1 | 4.1 | −156.8 | 41.5 | 0.8 | 0.7 | −17.6 | −16.5 | pET28b | 0 | 2 |
| DHF103 | 1 | 3.9 | −157.1 | 47.2 | 0.8 | 0.7 | −23.1 | −12.0 | pET28b | 0 | 2 |
| DHF104 | 1 | 8.3 | 131.5 | 27.5 | 0.8 | 0.6 | −18.9 | −9.7 | pET28b | 0 | 2 |
| DHF105 | 1 | 8.4 | 132.7 | 27.5 | 0.7 | 0.7 | −15.3 | −1.6 | pET28b | 0 | 2 |
| DHF106 | 1 | 8.4 | 131.9 | 29.4 | 0.8 | 0.7 | −16.9 | −12.9 | pET28b | 0 | 2 |
| DHF107 | 3 | 11.0 | 40.9 | 69.1 | 0.8 | 0.7 | −20.7 | −17.7 | pET28b | 1 | 2 |
| DHF108 | 1 | 12.0 | −149.2 | 24.9 | 0.8 | 0.6 | −25.8 | −20.7 | pET28b | 0 | 2 |
| DHF109 | 1 | 11.8 | −150 | 24.8 | 0.8 | 0.8 | −24.6 | −21.5 | pET28b | 0 | 0 |
| DHF110 | 1 | 13.3 | −152.6 | 23.0 | 0.8 | 0.7 | −14.0 | −11.5 | pET28b | 0 | 2 |
| DHF111 | 1 | 5.1 | −138.5 | 36.5 | 0.8 | 0.7 | −15.9 | −0.9 | pET28b | 0 | 2 |
| DHF112 | 1 | 6.4 | −145.3 | 30.4 | 0.8 | 0.7 | −17.3 | −9.3 | pET28b | 0 | 2 |
| DHF113 | 1 | 12.6 | −148.3 | 24.5 | 0.8 | 0.8 | −23.3 | −15.7 | pET28b | 0 | 2 |
| DHF114 | 1 | 12.4 | −146.5 | 24.8 | 0.8 | 0.7 | −25.8 | −0.6 | pET28b | 0 | 2 |
| DHF115 | 1 | 8.8 | −150.3 | 29.9 | 0.7 | 0.7 | −12.6 | −6.6 | pET28b | 0 | 2 |
| DHF116 | 1 | 14.8 | −152.9 | 19.0 | 0.7 | 0.7 | −31.7 | −8.3 | pET28b | 0 | 2 |
| DHF117 | 2 | 8.1 | −42 | 42.6 | 0.7 | 0.7 | −16.5 | −7.8 | pET28b | 0 | 2 |
| DHF118 | 1 | 5.3 | 107.3 | 48.4 | 0.8 | 0.7 | −15.3 | −9.9 | pET28b | 0 | 2 |
| DHF119 | 1 | 4.5 | −106.4 | 46.4 | 0.7 | 0.7 | −11.9 | −9.0 | pET28b | 1 | 2 |
| DHF120 | 1 | 8.6 | −110.6 | 27.5 | 0.7 | 0.7 | −19.6 | −16.3 | pET28b | 0 | 2 |
| DHF121 | 1 | 9.2 | −107 | 28.9 | 0.9 | 0.7 | −15.6 | −2.4 | pET28b | 0 | 2 |
| DHF122 | 1 | 4.6 | −81.3 | 43.4 | 0.8 | 0.7 | −29.7 | −9.3 | pET28b | 0 | 2 |
| DHF123 | 2 | 9.6 | 50.1 | 38.7 | 0.8 | 0.8 | −17.9 | −13.7 | pET28b | 0 | 0 |
| DHF124 | 1 | 12.3 | −147.1 | 24.7 | 0.8 | 0.7 | −30.4 | −16.3 | pET28b | 0 | 0 |

^{¶}Shape complementarity of main interface
^{§}Shape complementarity of secondary interface
^{†}Computed Binding energy of main interface
^{‡}Computed Binding energy of secondary interface
*0: no filaments observed by negative stain EM 1: filaments observed by EM
^{#}no expression, 1: insoluble expression 2: soluble expression

TABLE 2
CryoEM data collection and refinement

| | DHF119 | DHF38 | DHF58 | DHF46 | DHF79 | DHF91 |
|---|---|---|---|---|---|---|
| | (EMD-9021, | (EMD-9020, | (EMD-9017, | (EMD-9016, | (EMD-9018, | (EMD-9019, |
| | PDB 6E9Z) | PDB 6E9Y) | PDB 6E9T) | PDB 6E9R) | PDB 6E9V) | PDB 6E9X) |
| Data collection | | | | | | |
| Microscope | Titan Krios | Titan Krios | TF20 | TF20 | TF20 | TF20 |
| Voltage | 300 kV | 300 kV | 200 kV | 200 kV | 200 kV | 200 kV |
| Electron detector | K2 Summit | K2 Summit | K2 Summit | K2 Summit | K2 Summit | K2 Summit |
| Electron dose (e⁻/Å²) | 90 | 90 | 45 | 45 | 45 | 45 |
| Pixel size (Å) | 1.05 | 1.05 | 1.26 | 1.26 | 1.26 | 1.26 |
| Reconstruction | | | | | | |
| Point group | C3 | Cl | C2 | C4 | Cl | C3 |
| symmetry | | | | | | |
| Refined helical | 43.5023 | −88.17 | 40.93287 | −51.23024 | 77.6764 | 50.1109 |
| symmetry (twist) | | | | | | |
| (deg) | | | | | | |
| Refined helical | 14.3312 | 8.29531 | 9.10835 | 21.632 | 5.07952 | 24.1338 |
| symmetry (rise) | | | | | | |
| (Å) | | | | | | |
| Particles | 63,067 | 112,593 | 76,994 | 36,692 | 32,583 | 15,670 |
| Resolution (0.143 | 3.4 | 4.3 | 5.4 | 5.9 | 6.9 | 7.8 |
| fsc)(Å) | | | | | | |
| Reconstruction | Relion | Relion, | SPIDER | SPIDER | Relion | Relion |
| software | | Frealign | | | | |

1. A non-naturally occurring polypeptide comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 21, 1-20, 22-33, and 36, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is capable of end-to-end homo-polymerization.

2. The polypeptide of claim 1, wherein the polypeptide includes at least 80%, 85%, 90%, 91%, 92%, 93%, 94%, 95%, 96%, 97%, 98%, 99%, or 100% of the identified interface residues.

3. The polypeptide of claim 1, comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:8, 10, 14, and 19-21

4. The polypeptide of claim 1, wherein amino acid substitutions relative to the reference amino acid sequence are conservative amino acid substitutions.

5. A nucleic acid encoding the polypeptide of claim 1.

6. An expression vector comprising the nucleic acid of claim 5 operatively linked to a promoter.

7. A recombinant host cell comprising the nucleic acid of claim 5 and/or the expression vector of claim 6.

8. A homo-polymer comprising 2, 3, 4, 5, 10, 15, 20, 25, 50, 75, 100, or more identical polypeptides according to claim 1 associated end-to-end.

9. The homo-polymer of claim 8, wherein the homo-polymer comprises a helical filament.

10. The homo-polymer of claim 8, wherein the homo-polymer is bound to a surface.

11. The homo-polymer of claim 9, wherein the homo-polymer is bound to the surface via interaction with an anchor protein.

12. A method of making the homo-polymer of claim 8, comprising mixing multiple copies of a polypeptide comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 21, 1-20, 22-33, and 36, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is capable of end-to-end homo-polymerization, wherein the mixing occurs under conditions that promote homo-polymerization of the polypeptides.

13. The method of claim 12, wherein homo-polymerization at one or both ends of the homo-polymer is capped by mixing the polypeptides with a corresponding capping protein comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-33 and 36, wherein the polypeptide includes changes in at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is not capable of end-to-end homo-polymerization.

14. An anchor protein, comprising:
(a) an oligomeric protein of cyclic symmetry;
(b) an optional amino acid linker; and
(c) a polypeptide of claim 1 or a capping protein comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-33 and 36, wherein the polypeptide includes changes in at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is not capable of end-to-end homo-polymerization, linked covalently or non-covalently to the oligomeric protein of cyclic symmetry.

15. The anchor protein of claim 14, further comprising a fluorescent tag and/or one or more binding domains to direct the anchor protein to a desired location.

16. The anchor protein of claim 14, wherein the anchor protein comprises the amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the full length of the amino acid sequence selected from the group consisting of SEQ ID NO:34-35, wherein the polypeptide includes at least 50%, 55%, 60%, 65%, 70%, 75%, 80%, 85%, 90%, 91%, 92%, 93%, 94%, 95%, 96%, 97%, 98%, 99%, or 100% of the identified interface residues

17. A capping protein comprising an amino acid sequence that is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-33 and 36, wherein the polypeptide includes changes in at least 50%, 55%, 60%, 65%, 70%, 75%, 80%, 85%, 90%, 91%, 92%, 93%, 94%, 95%, 96%, 97%, 98%, 99%, or 100% of the identified interface residues, and wherein the polypeptide is not capable of end-to-end homo-polymerization.

18. The capping protein of claim 17, comprising an amino acid sequence is at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:37-40.

19. A nucleic acid encoding the protein of claim 14.

20.-21. (canceled)

22. A method for computational design of polypeptides capable of end-to-end homo-polymerization to form self-assembling helical filaments, comprising the steps described herein.


SEQUENCE LISTING (41 sequences)

SEQ ID NO: 1 - protein, 200 aa, Artificial Sequence
  Synthetic peptide
SSEKEELRERLVKICVELAKLKGDDTLKAAEAAEEAFRLVVLAAMLAGIDSSEVLELAIRLIKTCVVLAAMEGYDISEACRAAAEAFTRVAMAALRAGITSSLVLKAAIELIKECVLNAAVEGYDISEACRAAAEAFKRVAEAAKRAGITSLETLLRAIEEIRKRVEEAQREGNDISEACRQAAEEFRKKAEELKRRGDV

SEQ ID NO: 2 - protein, 199 aa, Artificial Sequence
  Synthetic peptide
MGLEKEVLRVKLVKICVLLARLKGDDTEEAREAARQAFEVVRLAAELAGIDSSEVLELAIRLIKECVENAQREGYDISLACMLAALAFKRVAEAAKRAGITSSEVLELAIRLIKECVENAQRDGYDISEACRAAAEAFKRVAEAAKRAGITSSETLKRAIEEIRKRVEEAQREGNEISEACRQAAEEFRKKAEELKRLE

SEQ ID NO: 3 - protein, 182 aa, Artificial Sequence
  Synthetic peptide
MGDSMKLVMLLLKKAVTLAKLNNDDMVALEIERAAKQIVLALAVNKSDEMAKVMLALAKAVLLAAKNNDDEVAREIARAAAEIVEALKENNSDEMAKVMLALAKAVLLAAKNNDDEVAREIARAAAEIVEALRENNSDEMAKKMLELAKRVLDAAKNNDDETAREIARQAAEEVEADRENLE

SEQ ID NO: 4 - protein, 211 aa, Artificial Sequence
  Synthetic peptide
  MISC_FEATURE (1)..(8): optional
MGHHHHHHSSGSSEKEELRERLVEIVVALAKAKGDDTELAREAAREAFELVREAAERAGIDSSMVLILAIQLILEVVANAAKEGYDISLAALAAAEAFKRVAEAAKRAGITSSMVLILAIRLIKEVVENAQREGYDISLAALAAAEAFKRVAEAAKRAGITSSMTLMRAIEEIRKRVEEAQREGNDISEAARQAAEEFIIKAVVLKARGDV

SEQ ID NO: 5 - protein, 199 aa, Artificial Sequence
  Synthetic peptide
MEKEVLREKLVKIVVEAAKLKGDDTLEAKLAAMEAYVLVVLAAELAGIDSSEVLELAIRLIKQVVILAALEGYDISEAARAAAEAFKRVAEAAKRAGITSSEVLELAIRLIKEVVENAQREGYDISEAARAAAEAFKRVAEAAKRAGITSSETLKRAIEEIRKRVEEAQREGNDISEAARQAAEEFRKKAEELKRRGLE

SEQ ID NO: 6 - protein, 200 aa, Artificial Sequence
  Synthetic peptide
MVEKNMLRLRLVKIVVENAKTKGDDTEEAREAAREAFEKVRVAAEVAGIDSSEVLELAIRLIKEVVLLALLEGYDISEAARAAAEAFVRVAVAAKIAGITSSEVLELAIRLIKEVVMNAMMEGYDISEAARAAAEAFKLVAEAAKRAGITSSETLKRAIEEIRKRVEEAQREGNNISVAALEAADEFRKKAEELKRRGLE

SEQ ID NO: 7 - protein, 180 aa, Artificial Sequence
  Synthetic peptide
  MISC_FEATURE (1)..(8): optional
MGHHHHHHSSGNDEKEKLKELLKRAEELAKSPDPEDLKEAVRLAEEVVRERPGSNLAKKALLIILRAAVELAALPDPEALKEAVKAAEKVVREQPGSSLARLALEIILMAAEELAKLPDPEALKEAVKAAEKVVREQPGSNLAKKALEIIERAAMMLKLSPDPEAQVEAMRAELKVVEER

SEQ ID NO: 8 - protein, 182 aa, Artificial Sequence
  Synthetic peptide
  MISC_FEATURE (1)..(8)
MGHHHHHHSSGTKEERVLLMKVAILAIVAAKKGNTDEVRKALELALLIAKVSGTTEAVKLALEVVARVAIEAARRGNTDAVREALEVALEIARESGTTEAVKLALEVVARVAIEAARRGNTEAVVEALLVALEIAKESGTEEAVRLALEVVKRVSNEALKQGNVDAVKVALEVRKMIEELSG

SEQ ID NO: 9 - protein, 171 aa, Artificial Sequence
  Synthetic peptide
SLKELLELERVAVEAIVAAAEGNTMEVIEQLQRAMLIAVLSGTTSAVKLALEVVARVAIEAARIGNTDAVRIALSVALTIAVISGTTEAVKLALEVVARVAIEAARRGNTDAVREALEVALEIARESGTEEAVRLALEVVKRVSDEAKKQGNEDAVKEAEEVRKKIEEESG

SEQ ID NO: 10 - protein, 168 aa, Artificial Sequence
  Synthetic peptide
MGDERRELEKVAVKAIMAAMLGNTDEVREQLQRALEIARESGTLLAVVLALEVVARVAIEAARKGNTDAVREALEVALEIARESGTKVAVVLALEVVARVAIEAARRGNVLAVILALEVALEIARESGTEEAALLAVEVVVRVSDEAKKQGNAVAVAVAEQVAKKILE

SEQ ID NO: 11 - protein, 227 aa, Artificial Sequence
  Synthetic peptide
ALDLLARVIVLVMEAVKLLVLAVIMGLEMILRVALRLAEEAARAAKAVLLLAEVEGDPEVALKAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREG

SEQ ID NO: 12 - protein, 229 aa, Artificial Sequence
  Synthetic peptide
MGLELSLVINLVLLANLLHLQAMMKGSSEDLEKALRTAEEAAREAKKVLEQAEKNGDPEVALRAVELVVIVAITLLLIAISSGSEEALERALRVAEEAARLAKRVLELAEKAGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGLE

SEQ ID NO: 13 - protein, 227 aa, Artificial Sequence
  Synthetic peptide
ARVVLKRVEKLVKEAETLLLVAIMKGSEEDLEKALRTAEEAAREAKKVLVLAVVVGDPEVALRAVELVVRVAELLLLIAIISGSEEALERALRVAEEAARLAKRVLELAENQGDPEVALRAVELVVRVAELLLMIAKVSGSEEALERALRVAEEAARLAKRVLELAEKQGDPEVARRAVELVKRVAELLERIARESGSEEAKERAERVREEALQLQARVMNLREREG

SEQ ID NO: 14 - protein, 227 aa, Artificial Sequence
  Synthetic peptide
AEELLKRVEKLVKEAEELLRQAMKKGSEELLEVALVVAQMAAREAKKVLTMAEVEGDPEVALRAVELVVRVAELLLRIALVSGSEEALERALRVAEEAARLAKRVLELAESQGDPEVALRAVELVVRVAELLLLIAKVSGSEEALERALRVAEEAARLAKRVLELAEKQGDPAVAILAVMLVKRVAELLENIARESGSEEAKERAERVREEARELQERVKELKERAG

SEQ ID NO: 15 - protein, 227 aa, Artificial Sequence
  Synthetic peptide
AVLALMVVNLLVQLAIELLSRAMLKGSAEDLEKALRTAEEAAREAKKVLEQAEKDGDPEVALLAVELVVVVATILLMIAKISGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREG

SEQ ID NO: 16 - protein, 230 aa, Artificial Sequence
  Synthetic peptide
MAVEEAIRVMRLVREAEQVLLQAKMMGSERVLEMALRTAEEAAREAKLVLAVAELEGDPVVALIAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGLE

SEQ ID NO: 17 - protein, 227 aa, Artificial Sequence
  Synthetic peptide
AIVQLMTVIAMAVIANALLIRAIVNGSAEDLEKALRTAEEAAREAKKVLEQAEKDGDPEVALRAVELVVLVAVNLLLIAKESGSEEALERALRVAEEAARLAKRVLELAEKVGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREG

SEQ ID NO: 18 - protein, 227 aa, Artificial Sequence
  Synthetic peptide
AVDQMIRVIVLVKEAEEALLNAKLLGSEMALTLALRTAEEAARVAKTVLELAEKDGDPVVALLAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREG

SEQ ID NO: 19 - protein, 227 aa, Artificial Sequence
  Synthetic peptide
MELLRVVMLVKEAEELLTLAVIKGSEDDLQKALRTAVEAAREAVKVLLQAVKRGDPEVALRAVELVVRVAELLLRIAKESGSELALKMALLVAEEAARLAKIVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGLE

SEQ ID NO: 20 - protein, 231 aa, Artificial Sequence
  Synthetic peptide
MGPEDELKRVEKLVKEAEMLLMLAKIKGSEKDLEKALRTAEEAAREAKKVLEQAEKEGDPEVALRAVELVVRVAELLLRIAKESGSEEALMTALLVAVVAATLAVRVLVLAAVQGDPEVALRAVELVVRVAELLLRIAKESGSEEALERALRVAEEAARLAKRVLELAQEQGDPVVAIAAVMLVKRVAELLERIARESGSEEAKERAERVREEARELQERVKELREREGLE

SEQ ID NO: 21 - protein, 231 aa, Artificial Sequence
  Synthetic peptide
MGPEDELKRVEKLVKEAEALLIVAKIKGSKRDLEKALRTAEEAAREAVKVLVQALLEGDPEVALRAVELVVRVAELLLRIAKESGSREALLRALIVAEEAAKLAKMVLELAEKQGDPEVALRAVELVVRVAELLLRIAKESGSEEALERALRVAEEAARLAKRVLELAEEQGDPLVAKMAVELVKRVAELLERIARESGSEEAKERAERVREEARELQERVKELREREGLE

SEQ ID NO: 22 - protein, 202 aa, Artificial Sequence
  Synthetic peptide
MGLILELAKLSLERARLASEAGDRVEFKIAAEKALLLAKVLVLQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVEEAAKVAEEVRKLAKKQGDEEVYEKARETAREVKEELKRVREEKGDLE

SEQ ID NO: 23 - protein, 201 aa, Artificial Sequence
  Synthetic peptide
MGQILMKAKLSLVKAAEASLSGDELEFRIAAETALVLAKLLVLQAKKKGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVEEAAKVAEEVRKLAKKQGDEEVYEKARETAREVKEELKRVREEKGLE

SEQ ID NO: 24 - protein, 199 aa, Artificial Sequence
  Synthetic peptide
AEVILLVAVLLLAKAVEAVLMGNRVAFRLAAELALRVAKLLVIMAKVEGDPELVLEAAKVALVVASLAALNGDKEVFKKAAESALEVAKRLVEVASKEGDPELVLEAAKVALKVAELAAKNGDKEVFKKAAESALEVAKRLVEVASKEGDPELVEEAAKVAEEVRKLAKKQGDEEVYEKARETAREVKEELKRVREEKG

SEQ ID NO: 25 - protein, 202 aa, Artificial Sequence
  Synthetic peptide
MGELVLLVAKLALAVAVQASALGDEEVFRKAAEKALELAKRLVELAKLIGDPELVLEAAKVALEVAELAAKNGDKEVFKKAAESALEVAKRLVEVASKEGDPELVLEAAKVALRVAELAAKNGDKEVFKKAAESALEVAKRLVEVASKEGDPELVEEAAKVAEEVRKLAKKQGDEEVYEKARETAREVKEELKRVREEKGLE

SEQ ID NO: 26 - protein, 203 aa, Artificial Sequence
  Synthetic peptide
MGPEEILESAKESLERAREASEAGLELVFRLAAEVALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPTLVIMALIVAEEVIKLASKQGDELVMLKAVETAKEVKEEAMRVMLEKGLE

SEQ ID NO: 27 - protein, 203 aa, Artificial Sequence
  Synthetic peptide
MGPEEILERAKESLERAREASERGDEMEFRIAAQKALELARQLVLQALAEGDPELVLEAAKVALRVAELAAKNGDKEVFKKAAESALEVAKVLVLVASKEGKPELVLEAAKVALRVAVLAAMNGDKEVFKKAAESALEVAKRLVEVASKEGDPELVLEAAKVAEKVRVLAAVQGDEEVYEKARETAREVKEELKRVMEEKGLE

SEQ ID NO: 28 - protein, 219 aa, Artificial Sequence
  Synthetic peptide
MQLMTAILMAILVATMVELVSNRALLEGNPDLVVSANELRRAVEEAIEEAKKQGNPELVMWVAIAAKVAAEVIKVAIQAELEGNRDLFRAALELVRAVIEAIEEAVKQGNPELVEWVARAAKVAAEVIKVAIQAEKEGNRDLFRAALELVRAVIEAIEEAVKQGNPELVERVARLAKKAAELIKRAIRAEKEGNRDERREALERVREVIERIEELVRQG

SEQ ID NO: 29 - protein, 220 aa, Artificial Sequence
  Synthetic peptide
INLTIAILVAILVATMVELVAIRAQLEGNPELAASAMELRRAVEEAIEEAEKQGNPLLVLWVAVAAKVAAEVIKVAIQAEKEGNRDLFRAALELVRAVIEAIEEAVKQGNPELVEWVARAAKVAAEVIKVAIQAEKEGNRDLFRAALELVRAVIEAIEEAVKQGNPELVERVARLAKKAAELIKRAIRAEKEGNRDERREALERVREVIERIEELVRQGN

SEQ ID NO: 30 - protein, 221 aa, Artificial Sequence
  Synthetic peptide
ADEELARILIEMAKTAAEAAQMAAEVTGDPRVRLLAVRLKMLALMAALEVILDPSSSDVNEALKLIVEAIEAAVKALLAAVRTGDPEVRELARELVRLAVEAAEEVQRNPSSSDVNEALKLIVEAIEAAVRALEAAERTGDPEVRELARELVRLAVEAAEEVQRNPSSEEVNEALKKIVKAIQEAVESLREAEESGDPEKREKARERVREAVERAEEVQRN

SEQ ID NO: 31 - protein, 221 aa, Artificial Sequence
  Synthetic peptide
MDEMKARLLIERAKEAAERAQEAAERTGDPLVRLLAEMLKRLAQEAAEEVKRDPSSSDVNEALKLIVEAIEAAVRALEAAERTGDPEVRELASELVRLAVEAAEEVQRNPSSSDVNEALKLIVEAIEAAVRALEAAERTGDPRVRELARELVRLAVEAAEEVQRNPSSVEVALALLKIVKAIEEAVESLREAEESGDPMKRLEAALRVREAVERAEEVQRN

SEQ ID NO: 32 - protein, 232 aa, Artificial Sequence
  Synthetic peptide
  MISC_FEATURE (1)..(8): optional
MGHHHHHHSSGSDEEEARELIERAAEAAKRAVEAAERTGDPNVVELAKELVRLAQEAAEEVKRDPSSSDVNEALKLIVEAIEAAVRALEAAERTGDPVVRLLARELVRLAVEAAEEVQRNPSSSDVNEALKLIVEAIEAAVRALEAAERTGDPEVRELARELVRLAVEAAEEVQRNPSSEQVNEALKKIVKAIQEAVESLREAEESGDPEKRDKARLRVLEAVIRAMVVQIN

SEQ ID NO: 33 - protein, 230 aa, Artificial Sequence
  Synthetic peptide
MGIVLVIESLEAEVRLEKAKVLSVLARVRGDLKELAEALIEEARAVQELARVACEKGNSEEAERASEKAQRVLEEARKVSEEAREQGDDEVLALALIAIALAVLALAEVACCRGNSEEAERASEKAQRVLEEARKVSEEAREQGDDEVLALALIAIALAVLALAEVACCRGNKEEAERAYEDARRVEEEARKVKESAEEQGDSEVKRLAEEAEQLAREARRHVQECRGLE

SEQ ID NO: 34 - protein, 343 aa, Artificial Sequence
  Synthetic peptide
MGGGLNDIFEAQKIEWHEGGSGGSGGSGGSTETLIRLLEELARVLLEILKQNGVPTNVIEAVRKAMEILLKMLKNSDNTAEAAAYMAIAMILLLILAKGGSGGSGGSGGSGGSSPEDELKRVEKLVKEAEALLIVAKIKGSKRDLEKALRTAEEAAREAKKVLEQAEKEGDPEVALRAVELVVRVAELLLRIAKESGSREALLRALIVAEEAARLAKRVLELAEKQGDPEVALRAVELVVRVAELLLRIAKESGSEEALERALRVAEEAARLAKRVLELAEEQGDPLVAKMAVELVKRVAELLERIARESGSEEAKERAERVREEARELQERVKELREREGLE

SEQ ID NO: 35 - protein, 592 aa, Artificial Sequence
  Synthetic peptide
  MISC_FEATURE (342)..(592): GFP
MGGGLNDIFEAQKIEWHEGGSGGSGGSGGSTETLIRLLEELARVLLEILKQNGVPTNVIEAVRKAMEILLKMLKNSDNTAEAAAYMAIAMILLLILAKGGSGGSGGSGGSGGSSPEDELKRVEKLVKEAEALLIVAKIKGSKRDLEKALRTAEEAAREAVKVLVQALLEGDPEVALRAVELVVRVAELLLRIAKESGSREALLRALIVAEEAAKLAKMVLELAEKQGDPEVALRAVELVVRVAELLLRIAKESGSEEALERALRVAEEAARLAKRVLELAEEQGDPLVAKMAVELVKRVAELLERIARESGSEEAKERAERVREEARELQERVKELREREGSGSGSGSGSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGSLE

SEQ ID NO: 36 - protein, 227 aa, Artificial Sequence
  Synthetic peptide
AAIETLNVMTLVMLALLLLIQAISKGSAEDLEKALRTAEEAAREAKKVLEQAEKEGDPDQALKAVELVVVVAQLLLQIAIASGSREALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREG

SEQ ID NO: 37 - protein, 231 aa, Artificial Sequence
  Synthetic peptide
MGPEDELKRVEKLVKEAEELLTLAVIKGSEDDLQKALRTAVEAAREAKKVLEQAEKEGDPEVALRAVELVVRVAELLLRIAKESGSELALKMALLVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGLE

SEQ ID NO: 38 - protein, 228 aa, Artificial Sequence
  Synthetic peptide
MGELLRVVMLVKEAEELLRQAKEKGSEEDLEKALRTAEEAAREAVKVLLQAVKRGDPEVALRAVELVVRVAELLLRIAKESGSEEALERALRVAEEAARLAKIVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGLE

SEQ ID NO: 39 - protein, 231 aa, Artificial Sequence
  Synthetic peptide
MGPEDELKRVEKLVKEAEELLRQAKEKGSEEDLEKALRTAEEAAREAVKVLVQALLEGDPEVALRAVELVVRVAELLLRIAKESGSREALLRALIVAEEAAKLAKMVLELAEKQGDPEVALRAVELVVRVAELLLRIAKESGSEEALERALRVAEEAARLAKRVLELAEEQGDPEVARRAVELVKRVAELLERIARESGSEEAKERAERVREEARELQERVKELREREGLE

SEQ ID NO: 40 - protein, 231 aa, Artificial Sequence
  Synthetic peptide
MGPEDELKRVEKLVKEAEALLIVAKIKGSKRDLEKALRTAEEAAREAKKVLEQAEKEGDPEVALRAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPLVAKMAVELVKRVAELLERIARESGSEEAKERAERVREEARELQERVKELREREGLE

SEQ ID NO: 41 - protein, 15 aa, Artificial Sequence
  Synthetic peptide
GLNDIFEAQKIEWHE